(19) United States
(12) Patent Application Publication
(10) Pub. No.: US 2022/0322919 A1
(43) Pub. Date: Oct. 13, 2022

(54) MEDICAL SUPPORT ARM AND MEDICAL SYSTEM

(71) Applicant: SONY GROUP CORPORATION, TOKYO (JP)

(72) Inventor: DAISUKE NAGAO, TOKYO (JP)

(21) Appl. No.: 17/640,702

(22) PCT Filed: Aug. 7, 2020
(86) PCT No.: PCT/JP2020/030299

§ 371 Date: Mar. 4, 2022

(30) Foreign Application Priority Data
Sep. 12, 2019 (JP) 2019-165851

(51) Int. Cl. A61B 1/00; A61B 34/32; A61B 1/313; A61B 17/08

(52) U.S. Cl. CPC A61B 1/00149; A61B 34/32; A61B 1/3132; A61B 17/083; A61B 2034/301

(57) ABSTRACT

A medical support arm includes: a support arm that supports an endoscope; an arm control unit that is configured to cause the support arm to perform a plurality of different interference avoidance operations for avoiding an interference between the endoscope and a surgical tool while maintaining a state in which an objective lens of the endoscope is directed to an observation target; and a determination unit that determines a combination of operation amounts of the plurality of interference avoidance operations.

### IELD

The present disclosure relates to a medical support arm and a medical system.

### BACKGROUND

In endoscopic surgery, an image of the abdominal cavity of a patient is captured using an endoscope such as an oblique-viewing endoscope, and surgery is performed while a captured image captured by the endoscope is displayed on a display.

For example, Patent Literature 1 discloses a technology related to controlling a degree of insertion of an oblique-viewing endoscope into a human body and a posture of the oblique-viewing endoscope.

### CITATION LIST

### Patent Literature

Patent Literature 1: JP 2016-219521 A

### SUMMARY

### Technical Problem

In laparoscopic surgery, a surgical tool is inserted into a body separately from an endoscope. In this case, it is desirable that a support arm supporting the endoscope moves the endoscope so as to avoid interference with the surgical tool so that an operator can appropriately perform the surgery. On the other hand, it is also necessary to move the endoscope so that the operator can easily see an observation target (for example, a site to be treated by the operator). Therefore, it is not easy to control the support arm so that the endoscope maintains a state suitable for surgery.

Therefore, the present disclosure proposes a medical support arm and a medical system capable of appropriately controlling movement of a support arm.

### Solution to Problem

To solve the above problem, a medical support arm according to the present disclosure includes: a support arm that supports an endoscope; an arm control unit that is configured to cause the support arm to perform a plurality of different interference avoidance operations for avoiding an interference between the endoscope and a surgical tool while maintaining a state in which an objective lens of the endoscope is directed to an observation target; and a determination unit that determines a combination of operation amounts of the plurality of interference avoidance operations.

### BRIEF DESCRIPTION OF DRAWINGS

FIG. 1 is a diagram illustrating a configuration of a robot arm that supports an endoscope.

FIG. 2 is a diagram illustrating an appearance of an oblique-viewing endoscope.

FIG. 3 is a diagram illustrating a three-dimensional surface conically spreading with respect to an observation point.

FIG. 4 is a diagram for describing an interference avoidance area.

FIG. 5 is a diagram illustrating the three-dimensional surface conically spreading with respect to the observation point and the columnar interference avoidance area in an overlapping manner.

FIG. 6 is an enlarged diagram of an area in the vicinity of a current position of the oblique-viewing endoscope.

FIG. 7 is a diagram illustrating an example of a program diagram designed in advance.

FIG. 8 is a diagram illustrating an example of a schematic configuration of an endoscopic surgery system to which the technology according to the present disclosure can be applied.

FIG. 9 is a block diagram illustrating an example of functional configurations of a camera head and a camera control unit (CCU) illustrated in FIG. 8.

FIG. 10 is a schematic diagram illustrating an appearance of a support arm device according to the present embodiment.

FIG. 11 is a schematic diagram illustrating a configuration of an oblique-viewing endoscope according to an embodiment of the present disclosure.

FIG. 12 is a schematic diagram illustrating the oblique-viewing endoscope and a forward-viewing endoscope in comparison.

FIG. 13 is a block diagram illustrating an example of a configuration of a medical observation system according to an embodiment of the present disclosure.

FIG. 14 is a diagram illustrating a specific configuration example of a robot arm device according to an embodiment of the present disclosure.

FIG. 15 is a flowchart illustrating an example of interference avoidance processing for avoiding an interference between the oblique-viewing endoscope and a surgical tool.

FIG. 16 is a diagram illustrating a modification of the oblique-viewing endoscope.

### DESCRIPTION OF EMBODIMENTS

Hereinafter, embodiments of the present disclosure will be described in detail with reference to the drawings. Note that, in each of the following embodiments, the same reference signs denote the same portions, and an overlapping description will be omitted.

Further, the present disclosure will be described in the following order.

1. Introduction

1-1. Purpose and the like of Present Embodiment

1-2. Outline of Present Embodiment

2. Configuration of Medical System

2-1. First Configuration Example (Endoscope System)

2-2. Specific Configuration Example of Support Arm Device

2-3. Specific Configuration Example of Endoscope

2-4. Second Configuration Example (Medical Observation System)

3. Operation of Medical System

4. Modification

5. Conclusion

<<1. Introduction>>

<1-1. Purpose and the Like of Present Embodiment>

In minimally invasive surgery such as laparoscopic surgery, an assistant called a scopist usually holds and operates an endoscope by hand according to an instruction of a surgeon or a procedure of surgery. The skill of the scopist allows the surgeon to see what he/she wants to see through an image captured by the endoscope.

In recent years, in surgery using the endoscope, a method in which an endoscope holder arm is substituted for the scopist has been proposed. However, this method has a problem that an operation method is complicated. In order to solve the problem of operability, the holder arm (hereinafter, referred to as a support arm) itself may autonomously moves the endoscope.

Note that, in minimally invasive surgery, an oblique-viewing endoscope, a side-viewing endoscope, or the like is used as the endoscope, and there is also a rigid endoscope having a variable oblique angle. In addition, there is also a rigid endoscope having a configuration in which a distal end portion can be bent. These rigid endoscopes have various advantages such as being able to observe an affected part in different directions or to observe an affected part without an interference with other surgical instruments in the body.

Conventionally, the scopist has avoided an interference between the oblique-viewing endoscope and the surgical instrument by adjusting a rotation amount and a degree of insertion/removal of the oblique-viewing endoscope based on experience. Note that the interference avoidance using the adjustment of the rotation amount has a disadvantage that an observation direction is changed. On the other hand, the interference avoidance using the adjustment of the degree of insertion/removal has a disadvantage that details of an observation target are lost. For this reason, the scopist instinctively combines two handling amounts (the rotation amount and the degree of insertion/removal), thereby achieving capturing of an optimal image desired by the surgeon while avoiding the interference between the oblique-viewing endoscope and the instrument.

In order to cause the support arm of the endoscope to perform such an operation, it is necessary for a control device (for example, a processor) that controls the support arm to autonomously determine each of the two handling amounts (the rotation amount and the degree of insertion/removal) without depending on human sense. However, such a determination method has not been implemented so far.

For example, Patent Literature 1 (JP 2016 -219521 A) discloses a technology related to controlling the degree of insertion and the posture of the oblique-viewing endoscope, but the technology described in Patent Literature 1 is not a model considering rotation of the oblique-viewing endoscope.

Therefore, in the present embodiment, a reference called a rotation-insertion ratio (R/I ratio) is defined, such that it is possible for a designer to design each of the rotation amount and the degree of insertion of the oblique-viewing endoscope depending on a situation. Then, in the present embodiment, the control device of the support arm operates the support arm by using the design result depending on the situation. As a result, an optimal image desired by the surgeon can be captured while avoiding the interference between the oblique-viewing endoscope and the instrument.

Note that, in the following description, “insertion” may be used as insertion in a broad sense including removal (pulling operation). The term “insertion” appearing in the following description can be replaced with “removal” or “insertion/removal” as appropriate. In addition, the term “insertion/removal” appearing in the following description can be replaced with “insertion” or “removal” as appropriate. Similarly, the term “removal” appearing in the following description can be replaced with “insertion” or “insertion/removal” as appropriate.

<1-2. Overview of Present Embodiment>

An operation for avoiding the interference between the oblique-viewing endoscope and the surgical tool (hereinafter, referred to as an interference avoidance operation) is determined by a combination of an operation of pulling the oblique-viewing endoscope (removal operation) and an operation of rotating the oblique-viewing endoscope (rotation operation). However, as described above, the rotation operation results in a change in observation direction and the removal operation results in a loss of details. Therefore, the control device of the support arm does not simply move the oblique-viewing endoscope in a certain constant direction (for example, a direction in which the oblique-viewing endoscope is pulled) determined in advance in order to avoid the interference.

In the present embodiment, the control device of the support arm calculates a ratio between a minimum operation amount of the support arm in a case where the oblique-viewing endoscope is pulled until the interference is eliminated and a minimum operation amount of the support arm in a case where the oblique-viewing endoscope is rotated until the interference is eliminated. Then, the control device determines a combined operation amount of the two operations (the removal operation and the rotation operation) on the basis of the ratio and information of a program diagram designed in advance. The ratio and the program diagram will be described in detail later.

Note that the operation amount can also be referred to as a handling amount. The term “operation amount” appearing in the following description can be replaced with the term “handling amount” as appropriate.

A method of determining the operation amount according to the present embodiment is a method of determining the operation amount according to the program diagram. Therefore, the designer of the control device can design a plurality of program diagrams in advance such that the control device of the support arm can change an adjustment method for the rotation operation and the removal operation according to a phase of the surgery. The control device of the support arm can perform an appropriate interference avoidance operation according to the phase of the surgery by using the information of the program diagram designed in advance.

For easy understanding, an outline of the present embodiment will be described below with reference to the drawings.

(Outline of Configuration of Device)

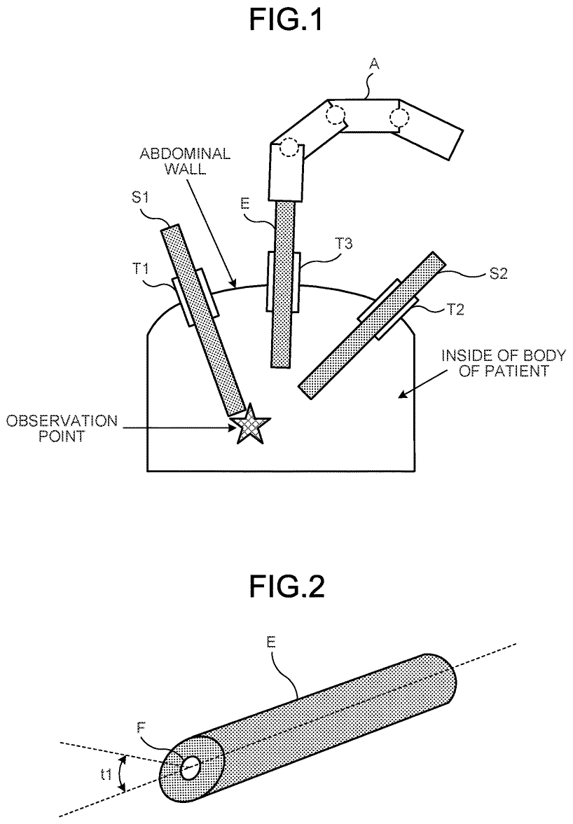

FIG. 1 is a diagram illustrating a configuration of a robot arm A (one aspect of a computer-aided surgery system) that supports an oblique-viewing endoscope E. The robot arm A is an example of a medical support arm of the present embodiment. The oblique-viewing endoscope E is connected to the robot arm A. As described above, the oblique-viewing endoscope is a type of endoscope. Note that, in the present embodiment, the endoscope includes a scope (lens barrel) and a camera head, but the endoscope does not have to necessarily include the camera head. For example, only a portion corresponding to the scope (lens barrel) may be regarded as the endoscope. The robot arm of the present embodiment supports, for example, the camera head to which the scope (lens barrel) is attached.

A motor for controlling each joint is arranged inside the robot arm A. The oblique-viewing endoscope E is inserted into the body of a patient through a trocar T1, and captures an image of an object or a point (hereinafter, referred to as an observation target or an observation point) in which an operator is interested and surroundings thereof. Here, a trocar T3 is an instrument called a medical puncture instrument. Note that a surgical instrument (for example, instruments S1 and S2 illustrated in FIG. 1) is also inserted into the body of the patient through the trocar (for example, the trocars T1 and T2 illustrated in FIG. 1). The operator (for example, the surgeon) performs laparoscopic surgery while viewing the image captured by the endoscope E.

(Relationship between Oblique-Viewing Endoscope and Conical Surface)

FIG. 2 is a diagram illustrating an appearance of the oblique-viewing endoscope E. The oblique-viewing endoscope E is on an axis, and includes an objective lens F at a distal end on the axis. An orientation of the objective lens F toward the observation point is inclined by an angle t1 with respect to an axial direction of the oblique-viewing endoscope E. As an example, the angle t1 is 30° to 40°. In the following description, the angle t1 may be referred to as an oblique angle.

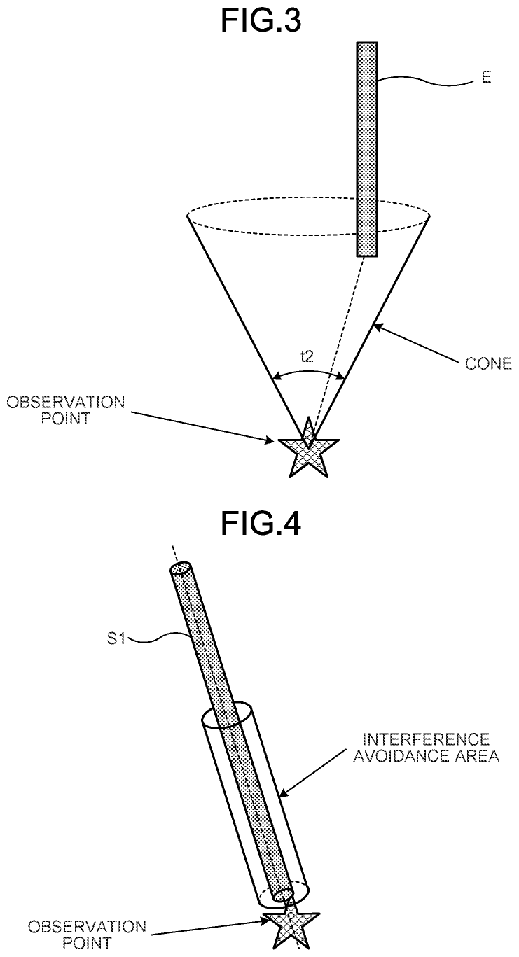

The oblique-viewing endoscope E can perform observation around the same point as long as a three-dimensional surface conically spreads with respect to the observation point. FIG. 3 is a diagram illustrating the three-dimensional surface conically spreading with respect to the observation point. A control device of the robot arm A can maintain a state in which the objective lens F faces the observation point by maintaining a position of the objective lens F of the oblique-viewing endoscope E on the conical surface. An angle t2 of an apex of this cone is determined according to the oblique angle t1.

(Setting of Interference Avoidance Area)

Note that, in the present embodiment, in order to avoid an interference between the oblique-viewing endoscope E and the surgical instrument, the control device of the robot arm A is operated so that the oblique-viewing endoscope E does not enter a column determined in advance according to the observation point. In the following description, this area for interference avoidance is referred to as an interference avoidance area.

FIG. 4 is a diagram for describing the interference avoidance area. In the example of FIG. 4, a columnar area having a predetermined radius centered on the surgical tool S1 is the interference avoidance area. A diameter of the column may be arbitrarily set according to the surgical tool. Note that the interference avoidance area is not necessarily columnar. For example, the interference avoidance area may have a shape in which a plurality of columns having different diameters are combined. In this case, the shape of the column may be changed depending on a distance to the observation point.

(Definition of R/I Ratio)

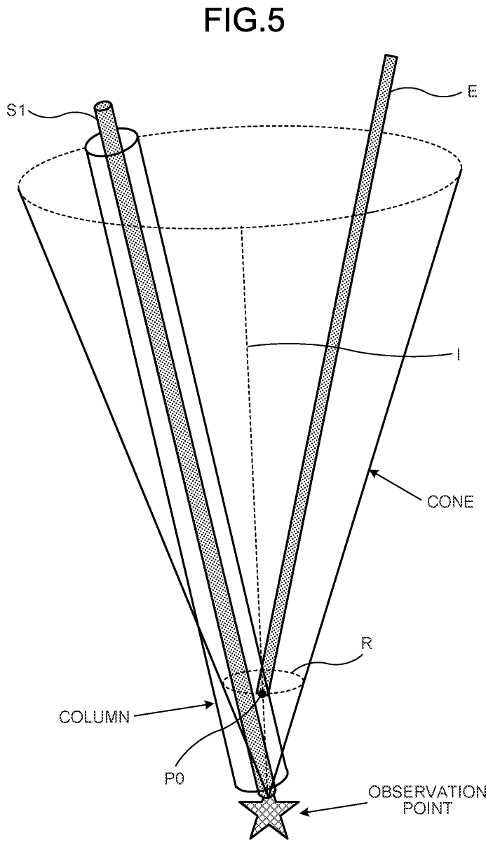

FIG. 5 is a diagram illustrating the three-dimensional surface conically spreading with respect to the observation point and the columnar interference avoidance area in an overlapping manner. In FIG. 5, a direction R indicates a direction (rotation direction) of the rotation operation of the oblique-viewing endoscope E, and a direction I indicates a direction (insertion/removal direction) of the insertion/removal operation (removal operation and insertion operation) of the oblique-viewing endoscope E. In addition, a point P0 indicates a current position of the objective lens F of the oblique-viewing endoscope E. The rotation direction R, the insertion/removal direction I, and the current position P0 are all positioned on the conical surface.

Note that, in the present embodiment, the rotation operation means that the objective lens F of the oblique-viewing endoscope E is moved in the rotation direction R along the conical surface, and the insertion/removal operation (removal operation and insertion operation) means that the objective lens F of the oblique-viewing endoscope E is moved in the rotation direction R along the conical surface.

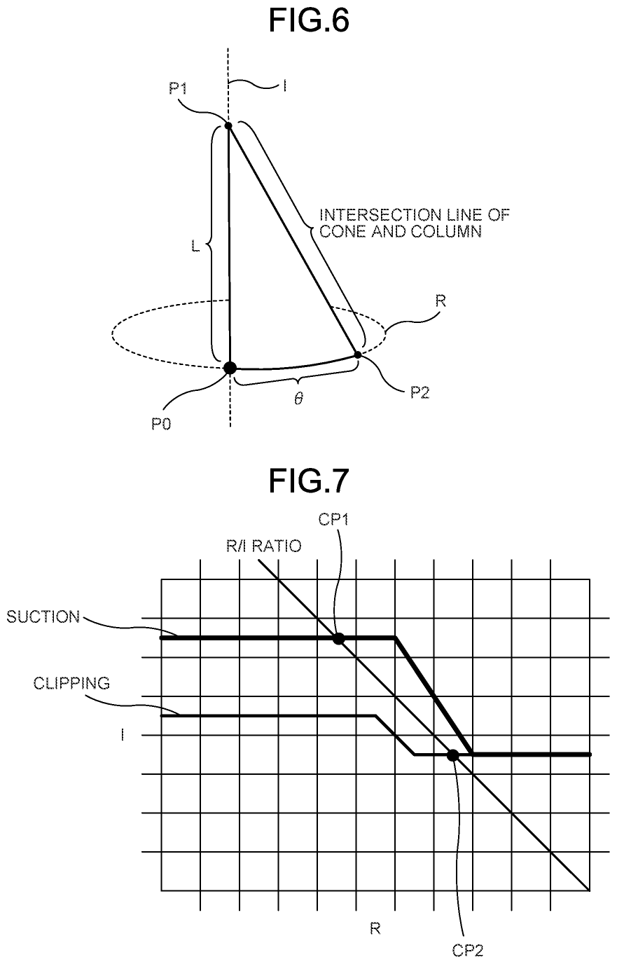

FIG. 6 is an enlarged diagram of an area in the vicinity of the current position P0 of the oblique-viewing endoscope E. An oblique line in FIG. 6 is an intersection line of surfaces of two solids (the cone and the column) in the vicinity of the current position P0. Here, the rotation-insertion ratio (R/I ratio) as shown in the following Equation (1) or the following Equation (2) is defined. The R/I ratio may be any one of Equation (1) and Equation (2).

R/I ratio=rθ/L (1)

R/I ratio=θ/L (2)

Here, θ is the minimum rotation amount with which the interference can be avoided only by the rotation operation from the current position P0. In addition, r is a radius of a circle formed by cutting the cone along the rotation direction so as to pass through the current position P. In addition, L is the minimum degree of insertion/removal with which the interference can be avoided only by the removal operation (pulling operation) from the current position P0. Note that the degree of insertion/removal can also be referred to as the degree of removal, the degree of insertion (negative degree of insertion), or the like.

A large R/I ratio indicates that the interference cannot be avoided unless the rotation amount is large, and a small R/I ratio indicates that the interference cannot be avoided unless the degree of insertion/removal is high.

Since Equation (1) is an equation in which the rotation angle θ and the radius r are taken into consideration, both the denominator and the numerator have the same distance unit. Therefore, in a case where Equation (2) is used for defining the R/I ratio, a highly accurate calculation result can be expected. However, it is necessary to calculate the radius r accordingly, which increases a processing load of the control device. On the other hand, Equation (2) is a simplified expression in which the radius r is omitted. Therefore, in a case where Equation (2) is used for defining the R/I ratio, the calculation load of the control device can be reduced although accuracy is slightly sacrificed. The control device (or the designer of the control device) may select whether to use Equation (1) or Equation (2) for the definition of the R/I ratio, in consideration of these advantages and disadvantages.

(Program Diagram)

The control device determines a combined operation amount of the two operations (the removal operation and the rotation operation) on the basis of the R/I ratio and the information of the program diagram designed in advance.

FIG. 7 is a diagram illustrating an example of the program diagram designed in advance. The program diagram illustrated in FIG. 7 is a graph with R on a horizontal axis and I on a vertical axis. Note that, in the following description, R may be used as a variable indicating the rotation amount, instead of a sign indicating the rotation direction. Further, in the following description, I may be used as a variable indicating the degree of insertion/removal (the degree of insertion or the degree of removal), instead of a sign indicating the insertion/removal direction (insertion direction or removal direction). In the program diagram illustrated in FIG. 7, the degree of removal increases upward, and the rotation amount increases rightward. Note that the rotation amount R on the horizontal axis may be in units of radius×rotation angle, or may be in units of rotation angle.

The control device of the robot arm A determines the degree of insertion/removal and the rotation amount indicated by an intersection of a line indicated by the calculated R/I ratio (hereinafter, also referred to as an oblique line) and a line designed in advance (hereinafter, also referred to as a designed line) as the combined operation amount of the oblique-viewing endoscope E. Here, the designed line is a line indicated by “suction” or “clipping” in the example of FIG. 7.

The R/I ratio has the same value at an arbitrary point on the oblique line. The control device of the robot arm A can achieve the interference avoidance by setting the values of R and I indicated by the arbitrary point on the oblique line as the combined operation amount (the degree of insertion/removal and the rotation amount). Note that the designer of the control device can design a plurality of designed lines such as lines indicated by “suction” and “clipping” illustrated in FIG. 7, according to the situation of the surgery. Here, the suction is a treatment of sucking liquid in the body by using a suction instrument, and the clipping is a treatment of clipping a blood vessel. Since the clipping is delicate work, an image having a high image quality is desired, whereas the suction does not necessarily require an image having a high image quality.

The designer of the control device designs the program diagram in consideration of these circumstances. For example, the designer performs design so that a change in degree of insertion/removal does not occur as much as possible such that the image quality is maintained at the time of performing the clipping requiring high precision. The designed line of the clipping illustrated in FIG. 7 is an example in which the design is performed so that a change in degree of insertion/removal does not occur as much as possible at the time of performing the clipping. On the other hand, the design is performed so that a relatively large change in degree of insertion is allowed at the time of performing the suction. The designed line of the suction illustrated in FIG. 7 is an example in which a relatively large change in degree of insertion/removal is allowed at the time of performing the suction.

Note that the program diagram may be designed by a computer instead of a person (designer). At this time, the computer may be the control device of the robot arm A or a computer (for example, a server device or a personal computer) for designing the program diagram independent of the robot arm A. The term “designer” appearing in the following description can be replaced with a computer (control device or design device).

The control device of the robot arm A determines the combined operation amount (the degree of insertion/removal and the rotation amount) on the basis of such a program diagram. For example, in a case where a treatment currently performed by the operator is “suction”, the control device sets, as the combined operation amount, values of the rotation amount (R) and the degree (I) of insertion/removal indicated by an intersection CP1 of the oblique line indicating the R/I ratio and the designed line indicating the suction. On the other hand, in a case where the treatment currently performed by the operator is “clipping”, the control device sets, as the combined operation amount, values of R and I indicated by an intersection CP2 of the oblique line indicating the R/I ratio and the designed line indicating the suction. The robot arm A can perform an appropriate interference avoidance operation according to the situation of the surgery by determining the combined operation amount on the basis of the program diagram.

Although the outline of the present embodiment has been described above, a medical system (computer-aided surgery system) including the medical support arm (for example, the robot arm A) of the present embodiment will be described in detail below.

<<2. Configuration of Medical System>>

Before describing an operation of the medical system of the present embodiment, a configuration (device configuration and functional configuration) of the medical system will be described. For the medical system of the present embodiment, several configuration examples can be considered.

<2-1. First Configuration Example (Endoscope System)>

First, a configuration of an endoscope system will be described as an example of the medical system of the present embodiment.

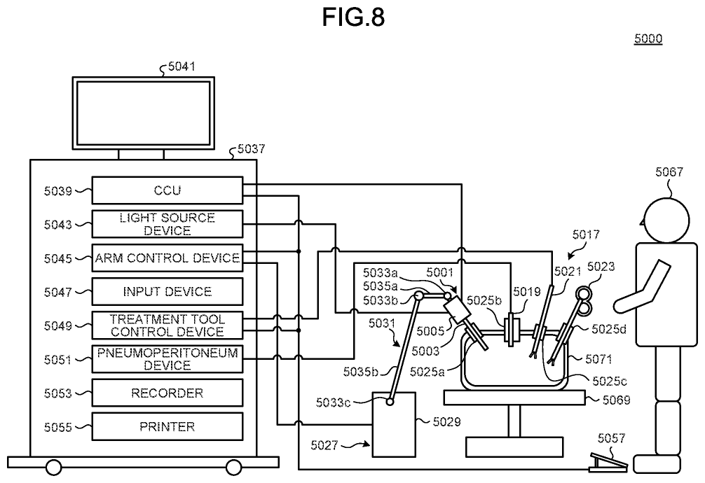

FIG. 8 is a diagram illustrating an example of a schematic configuration of an endoscopic surgery system 5000 to which the technology according to the present disclosure can be applied. In the example of FIG. 8, a state in which an operator (for example, a doctor) 5067 is performing surgery on a patient 5071 on a patient bed 5069 by using the endoscopic surgery system 5000 is illustrated. As illustrated, the endoscopic surgery system 5000 includes an endoscope 5001, other surgical tools 5017, a support arm device 5027 that supports the endoscope 5001, and a cart 5037 on which various devices for endoscopic surgery are mounted.

The endoscope 5001 corresponds to, for example, the endoscope E illustrated in FIGS. 1 to 3 and 5, and the support arm device 5027 corresponds to, for example, the robot arm A illustrated in FIG. 1.

In endoscopic surgery, instead of cutting and opening the abdominal wall, a plurality of cylindrical puncture instruments called trocars 5025a to 5025d puncture the abdominal wall. Then, a lens barrel 5003 of the endoscope 5001 and the other surgical tools 5017 are inserted into the body cavity of the patient 5071 through the trocars 5025a to 5025d. In the illustrated example, as the other surgical tools 5017, a pneumoperitoneum tube 5019, an energy treatment tool 5021, and forceps 5023 are inserted into the body cavity of the patient 5071. Furthermore, the energy treatment tool 5021 is a treatment tool for incision and peeling of tissue, vascular closure, or the like by using a high-frequency current or ultrasonic vibration. However, the illustrated surgical tools 5017 are merely an example, and various surgical tools generally used in endoscopic surgery, such as tweezers and a retractor may be used as the surgical tools 5017.

An image of a surgical site in the body cavity of the patient 5071 captured by the endoscope 5001 is displayed on a display device 5041. The operator 5067 performs treatment such as resection of an affected part by using the energy treatment tool 5021 or the forceps 5023 while viewing the image of the surgical site displayed on the display device 5041 in real time. Note that, although not illustrated, the pneumoperitoneum tube 5019, the energy treatment tool 5021, and the forceps 5023 are supported by the operator 5067, an assistant, or the like during surgery.

[Support Arm Device]

The support arm device 5027 includes an arm portion 5031 extending from a base portion 5029. In the illustrated example, the arm portion 5031 includes joint portions 5033a, 5033b, and 5033c and links 5035a and 5035b, and is driven under the control of an arm control device 5045. The arm portion 5031 supports the endoscope 5001 and controls a position and a posture of the endoscope 5001. As a result, it is possible to stably fix the position of the endoscope 5001.

[Endoscope]

The endoscope 5001 includes the lens barrel 5003 in which a region corresponding to a predetermined length from a distal end is inserted into the body cavity of the patient 5071, and a camera head 5005 connected to a proximal end of the lens barrel 5003. In the illustrated example, the endoscope 5001 configured as a so-called rigid endoscope including the rigid lens barrel 5003 is illustrated, but the endoscope 5001 may be configured as a so-called flexible endoscope including the flexible lens barrel 5003.

An opening portion into which an objective lens is fitted is provided at the distal end of the lens barrel 5003. A light source device 5043 is connected to the endoscope 5001, and light generated by the light source device 5043 is guided to the distal end of the lens barrel by a light guide extending inside the lens barrel 5003, and is emitted toward an observation target in the body cavity of the patient 5071 via the objective lens. Note that the endoscope 5001 may be a forward-viewing endoscope, an oblique-viewing endoscope, or a side-viewing endoscope.

An optical system and an imaging element are provided inside the camera head 5005, and reflected light (observation light) from the observation target is collected on the imaging element by the optical system. The observation light is photoelectrically converted by the imaging element, and an electric signal corresponding to the observation light, that is, an image signal corresponding to an observation image is generated. The image signal is transmitted to a camera control unit (CCU) 5039 as raw data. Note that the camera head 5005 has a function of adjusting a magnification and a focal length by appropriately driving the optical system.

Note that, for example, a plurality of imaging elements may be provided in the camera head 5005 in order to support stereoscopic viewing (3D display) or the like. In this case, a plurality of relay optical systems are provided inside the lens barrel 5003 in order to guide the observation light to each of the plurality of imaging elements.

[Various Devices Mounted on Cart]

The CCU 5039 is implemented by a central processing unit (CPU), a graphics processing unit (GPU), and the like, and integrally controls operations of the endoscope 5001 and the display device 5041. Specifically, the CCU 5039 performs, on the image signal received from the camera head 5005, various types of image processing for displaying an image based on the image signal, such as development processing (demosaic processing), for example. The CCU 5039 provides the image signal subjected to the image processing to the display device 5041. Furthermore, the CCU 5039 transmits a control signal to the camera head 5005 to control driving thereof. The control signal can include information regarding imaging conditions such as a magnification and a focal length.

The display device 5041 displays an image based on the image signal subjected to the image processing by the CCU 5039 under the control of the CCU 5039. In a case where the endoscope 5001 supports high-resolution imaging such as 4K (the number of horizontal pixels 3840× the number of vertical pixels 2160) or 8K (the number of horizontal pixels 7680× the number of vertical pixels 4320), and/or in a case where the endoscope supports 3D display, a display device capable of high-resolution display and/or a display device capable of 3D display can be used as the display device 5041 for each case. In a case where the display device supports the high-resolution imaging such as 4K or 8K, a further immersive feeling can be obtained by using, as the display device 5041, a display device with a size of 55 inches or more. Furthermore, a plurality of display devices 5041 having different resolutions and sizes may be provided depending on the application.

The light source device 5043 is implemented by a light source such as a light emitting diode (LED), for example, and supplies, to the endoscope 5001, irradiation light for capturing an image of the surgical site.

The arm control device 5045 is implemented by, for example, a processor such as a CPU, and is operated according to a predetermined program to control driving of the arm portion 5031 of the support arm device 5027 according to a predetermined control method. The arm control device 5045 corresponds to the control device (for example, the control device for the robot arm A) that controls the support arm of the present embodiment. Note that the CCU 5039 can also be regarded as the control device of the present embodiment.

An input device 5047 is an input interface for the endoscopic surgery system 5000. A user can input various types of information or instructions to the endoscopic surgery system 5000 via the input device 5047. For example, the user inputs various types of information regarding surgery, such as physical information of a patient and information regarding a surgical procedure of the surgery, via the input device 5047. Furthermore, for example, the user inputs an instruction to drive the arm portion 5031, an instruction to change the imaging conditions (a type of the irradiation light, a magnification, a focal length, and the like) of the endoscope 5001, an instruction to drive the energy treatment tool 5021, and the like via the input device 5047.

The type of the input device 5047 is not limited, and the input device 5047 may be various known input devices. As the input device 5047, for example, a mouse, a keyboard, a touch panel, a switch, a foot switch 5057, a lever, and/or the like can be applied. In a case where a touch panel is used as the input device 5047, the touch panel may be provided on a display surface of the display device 5041.

Alternatively, the input device 5047 is a device worn by the user, such as a glasses-type wearable device or a head-mounted display (HMD), and various inputs are performed according to a gesture or a gaze of the user detected by these devices. Furthermore, the input device 5047 includes a camera capable of detecting movement of the user, and various inputs are performed according to a gesture or a gaze of the user detected from a video captured by the camera. Furthermore, the input device 5047 includes a microphone capable of collecting user's voice, and various inputs are performed by voice via the microphone. As described above, the input device 5047 is configured to be able to input various types of information in a non-contact manner, and thus, in particular, a user (for example, the operator 5067) belonging to a clean area can operate a device belonging to an unclean area in a non-contact manner. In addition, since the user can operate the device without releasing his/her hand from the held surgical tool, the convenience of the user is improved.

A treatment tool control device 5049 controls driving of the energy treatment tool 5021 for cauterization and incision of tissue, vascular closure, or the like. A pneumoperitoneum device 5051 feeds gas into the body cavity of the patient 5071 via the pneumoperitoneum tube 5019 in order to inflate the body cavity for the purpose of securing a clear view for the endoscope 5001 and securing a working space for the operator. A recorder 5053 is a device capable of recording various types of information regarding surgery. A printer 5055 is a device capable of printing various types of information regarding surgery in various formats such as text, images, or graphs.

Hereinafter, a particularly characteristic configuration of the endoscopic surgery system 5000 will be described in more detail.

[Support Arm Device]

The support arm device 5027 includes the base portion 5029 which is a base, and the arm portion 5031 extending from the base portion 5029. The support arm device 5027 may include a control device that functions as the arm control device 5045 and/or the CCU 5039. The support arm device 5027 corresponds to the support arm (for example, the robot arm A) of the present embodiment. The arm portion 5031 may be regarded as the support arm of the present embodiment.

In the illustrated example, the arm portion 5031 includes the plurality of joint portions 5033a, 5033b, and 5033c and the plurality of links 5035a and 5035b connected by the joint portion 5033b, but in FIG. 8, the configuration of the arm portion 5031 is illustrated in a simplified manner for the sake of simplicity. In actual implementation, the shapes, the numbers, and the arrangements of the joint portions 5033a to 5033c and the links 5035a and 5035b, directions of rotation axes of the joint portions 5033a to 5033c, and the like can be appropriately set so that the arm portion 5031 has a desired degree of freedom. For example, the arm portion 5031 can be suitably configured to have six degrees of freedom or more. As a result, since the endoscope 5001 can be freely moved within a movable range of the arm portion 5031, the lens barrel 5003 of the endoscope 5001 can be inserted into the body cavity of the patient 5071 from a desired direction.

Actuators are provided in the joint portions 5033a to 5033c, and the joint portions 5033a to 5033c are configured to be rotatable around predetermined rotation axes by driving of the actuators. The driving of the actuator is controlled by the arm control device 5045, whereby a rotation angle of each of the joint portions 5033a to 5033c is controlled, and the driving of the arm portion 5031 is controlled. As a result, it is possible to control the position and the posture of the endoscope 5001. At this time, the arm control device 5045 can control the driving of the arm portion 5031 by various known control methods such as a power control or a position control.

For example, the operator 5067 may appropriately perform an operation input via the input device 5047 (including the foot switch 5057) to cause the arm control device 5045 to appropriately control the driving of the arm portion 5031 according to the operation input, thereby controlling the position and the posture of the endoscope 5001. With this control, the endoscope 5001 at the distal end of the arm portion 5031 can be moved from an arbitrary position to an arbitrary position and then fixedly supported at the position after the movement. Note that the arm portion 5031 may be operated by a so-called master-slave method. In this case, the arm portion 5031 (slave) can be remotely operated by the user via the input device 5047 (master console) installed at a place away from an operating room or in the operating room.

Furthermore, in a case where the power control is applied, the arm control device 5045 may perform a so-called power assist control of receiving an external force from the user and driving the actuator of each of the joint portions 5033a to 5033c so that the arm portion 5031 is smoothly moved according to the external force. As a result, when the user moves the arm portion 5031 while directly touching the arm portion 5031, the arm portion 5031 can be moved with a relatively small force. Therefore, it is possible to more intuitively move the endoscope 5001 with a simpler operation, and the convenience of the user can be improved.

Here, in general, in endoscopic surgery, the endoscope 5001 is supported by a doctor called scopist. However, the use of the support arm device 5027 enables more reliable fixation of the position of the endoscope 5001 without manual operation, and thus, it is possible to stably obtain the image of the surgical site and smoothly perform the surgery.

Note that the arm control device 5045 is not necessarily provided in the cart 5037. Furthermore, the arm control device 5045 is not necessarily one device. For example, the arm control device 5045 may be provided in each of the joint portions 5033a to 5033c of the arm portion 5031 of the support arm device 5027, and a driving control for the arm portion 5031 may be implemented by a plurality of arm control devices 5045 cooperating with each other.

[Light Source Device]

The light source device 5043 supplies the irradiation light for capturing an image of the surgical site to the endoscope 5001. The light source device 5043 includes, for example, a white light source implemented by an LED, a laser light source, or a combination thereof. At this time, in a case where the white light source is implemented by a combination of RGB laser light sources, an output intensity and an output timing of each color (each wavelength) can be controlled with high accuracy, and thus, white balance adjustment of the captured image can be performed in the light source device 5043. Furthermore, in this case, the observation target is irradiated with laser light from each of the RGB laser light sources in a time division manner and the driving of the imaging element of the camera head 5005 is controlled in synchronization with a timing of the irradiation, such that it is also possible to capture an image corresponding to each of RGB in a time division manner. With this method, a color image can be obtained without providing a color filter in the imaging element.

Furthermore, the driving of the light source device 5043 may be controlled so as to change the intensity of light to be output every predetermined time. The driving of the imaging element of the camera head 5005 is controlled in synchronization with a timing of the change of the intensity of the light to acquire images in a time division manner and images are combined, such that it is possible to generate a high-dynamic-range image without so-called underexposure and overexposure.

Furthermore, the light source device 5043 may be configured to be able to supply light in a predetermined wavelength band corresponding to special light observation. In the special light observation, for example, so-called narrow band imaging, in which an image of a predetermined tissue such as a blood vessel in a mucosal epithelial layer is captured with high contrast by radiating light in a narrower band than irradiation light (that is, white light) used at the time of normal observation, by using wavelength dependency of light absorption in a body tissue, is performed. Alternatively, in the special light observation, fluorescence observation for obtaining an image by fluorescence generated by irradiation with excitation light may be performed. In the fluorescence observation, for example, fluorescence from a body tissue can be observed by irradiating the body tissue with excitation light (autofluorescence observation), or a fluorescent image can be obtained by locally injecting a reagent such as indocyanine green (ICG) into a body tissue and irradiating the body tissue with excitation light corresponding to a fluorescence wavelength of the reagent. The light source device 5043 can be configured to be able to supply narrow band light and/or excitation light corresponding to such special light observation.

[Camera Head and CCU]

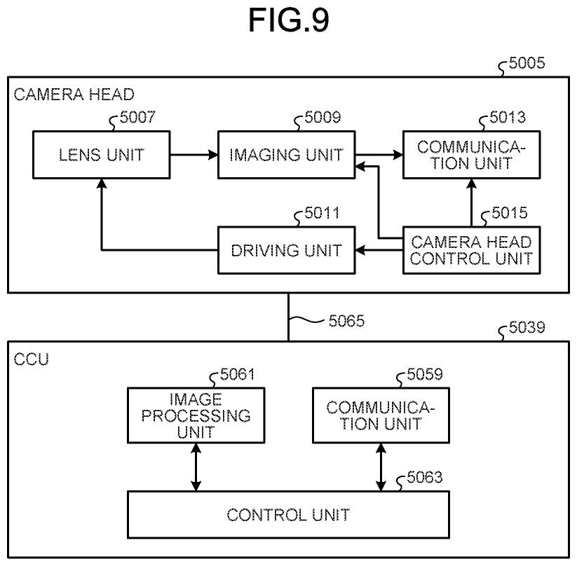

Functions of the camera head 5005 and the CCU 5039 of the endoscope 5001 will be described in more detail with reference to FIG. 9. FIG. 9 is a block diagram illustrating an example of functional configurations of the camera head 5005 and the CCU 5039 illustrated in FIG. 8.

Referring to FIG. 9, the camera head 5005 includes a lens unit 5007, an imaging unit 5009, a driving unit 5011, a communication unit 5013, and a camera head control unit 5015 as the functions thereof. Further, the CCU 5039 includes a communication unit 5059, an image processing unit 5061, and a control unit 5063 as the functions thereof. The camera head 5005 and the CCU 5039 are connected by a transmission cable 5065 so as to be bidirectionally communicable.

First, the functional configuration of the camera head 5005 will be described. The lens unit 5007 is an optical system provided at a portion at which the camera head 5005 is connected to the lens barrel 5003. The observation light taken in from the distal end of the lens barrel 5003 is guided to the camera head 5005 and is incident on the lens unit 5007. The lens unit 5007 is implemented by combining a plurality of lenses including a zoom lens and a focus lens. An optical characteristic of the lens unit 5007 is adjusted so as to concentrate the observation light on a light receiving surface of the imaging element of the imaging unit 5009. In addition, the zoom lens and the focus lens are configured to be movable on an optical axis thereof in order to adjust a magnification and a focal point of the captured image.

The imaging unit 5009 includes the imaging element and is arranged at a subsequent stage of the lens unit 5007. The observation light having passed through the lens unit 5007 is collected on the light receiving surface of the imaging element, and an image signal corresponding to the observation image is generated by photoelectric conversion. The image signal generated by the imaging unit 5009 is provided to the communication unit 5013.

For example, a complementary metal oxide semiconductor (CMOS) image sensor that has a Bayer array and is capable of color capturing is used as the imaging element included in the imaging unit 5009. Note that, as the imaging element, for example, an imaging element that can support the high-resolution imaging of 4K or more may be used. Since the high-resolution image of the surgical site is obtained, the operator 5067 can grasp a state of the surgical site in more detail, and can progress the surgery more smoothly.

Furthermore, the imaging element included in the imaging unit 5009 includes a pair of imaging elements for acquiring image signals for the right eye and the left eye corresponding to 3D display, respectively. As the 3D display is performed, the operator 5067 can more accurately grasp a depth of a living tissue in the surgical site. Note that, in a case where the imaging unit 5009 is configured as a multi-plate type, a plurality of lens units 5007 are provided corresponding to the respective imaging elements.

Furthermore, the imaging unit 5009 does not have to be necessarily provided in the camera head 5005. For example, the imaging unit 5009 may be provided immediately behind the objective lens inside the lens barrel 5003.

The driving unit 5011 is implemented by an actuator, and moves the zoom lens and the focus lens of the lens unit 5007 by a predetermined distance along the optical axis under the control of the camera head control unit 5015. As a result, the magnification and the focal point of the image captured by the imaging unit 5009 can be appropriately adjusted.

The communication unit 5013 is implemented by a communication device for transmitting and receiving various types of information to and from the CCU 5039. The communication unit 5013 transmits the image signal obtained from the imaging unit 5009 as raw data to the CCU 5039 via the transmission cable 5065. At this time, in order to display the captured image of the surgical site with low latency, the image signal is preferably transmitted by optical communication. This is because, at the time of surgery, the operator 5067 performs surgery while observing the state of the affected part in the captured image, and thus, for safer and more reliable surgery, it is required to display a moving image of the surgical site in real time as much as possible. In a case where optical communication is performed, a photoelectric conversion module that converts an electric signal into an optical signal is provided in the communication unit 5013. The image signal is converted into the optical signal by the photoelectric conversion module and then transmitted to the CCU 5039 via the transmission cable 5065.

Furthermore, the communication unit 5013 receives a control signal for controlling driving of the camera head 5005 from the CCU 5039. The control signal includes, for example, information regarding imaging conditions such as information for specifying a frame rate of the captured image, information for specifying an exposure value at the time of imaging, and/or information for specifying the magnification and the focal point of the captured image. The communication unit 5013 provides the received control signal to the camera head control unit 5015. Note that the control signal from the CCU 5039 may also be transmitted by optical communication. In this case, the photoelectric conversion module that converts an optical signal into an electric signal is provided in the communication unit 5013, and the control signal is converted into an electric signal by the photoelectric conversion module and then provided to the camera head control unit 5015.

Note that the imaging conditions such as the frame rate, the exposure value, the magnification, and the focal point are automatically set by the control unit 5063 of the CCU 5039 on the basis of the acquired image signal. That is, the endoscope 5001 has a so-called auto exposure (AE) function, an auto focus (AF) function, and an auto white balance (AWB) function.

The camera head control unit 5015 controls the driving of the camera head 5005 on the basis of the control signal received from the CCU 5039 via the communication unit 5013. For example, the camera head control unit 5015 controls driving of the imaging element of the imaging unit 5009 on the basis of the information for specifying the frame rate of the captured image and/or the information for specifying light exposure at the time of imaging. Furthermore, for example, the camera head control unit 5015 appropriately moves the zoom lens and the focus lens of the lens unit 5007 via the driving unit 5011 on the basis of the information for specifying the magnification and the focal point of the captured image. The camera head control unit 5015 may further have a function of storing information for identifying the lens barrel 5003 or the camera head 5005.

Note that, as the lens unit 5007, the imaging unit 5009, and the like are arranged in a sealed structure having high airtightness and waterproofness, the camera head 5005 can have resistance to autoclave sterilization processing.

Next, the functional configuration of the CCU 5039 will be described. The communication unit 5059 is implemented by a communication device for transmitting and receiving various types of information to and from the camera head 5005. The communication unit 5059 receives the image signal transmitted from the camera head 5005 via the transmission cable 5065. At this time, as described above, the image signal can be suitably transmitted by optical communication. In this case, for optical communication, a photoelectric conversion module that converts an optical signal into an electric signal is provided in the communication unit 5059. The communication unit 5059 provides the image signal converted into the electric signal to the image processing unit 5061.

Furthermore, the communication unit 5059 transmits a control signal for controlling the driving of the camera head 5005 to the camera head 5005. The control signal may also be transmitted by optical communication.

The image processing unit 5061 performs various types of image processing on the image signal that is raw data transmitted from the camera head 5005. Examples of the image processing include various types of known signal processing such as development processing, image quality enhancement processing (band emphasis processing, super-resolution processing, noise reduction (NR) processing, image stabilization processing, and/or the like), and/or enlargement processing (electronic zoom processing). Furthermore, the image processing unit 5061 performs wave detection processing on the image signal for performing the AE, the AF, and the AWB.

The image processing unit 5061 is implemented by a processor such as a CPU or a GPU, and the processor is operated according to a predetermined program, whereby the above-described image processing and wave detection processing can be performed. Note that, in a case where the image processing unit 5061 is implemented by a plurality of GPUs, the image processing unit 5061 appropriately divides information related to the image signal, and the plurality of GPUs perform the image processing in parallel.

The control unit 5063 performs various types of controls related to capturing of the image of the surgical site performed by the endoscope 5001 and display of the captured image. For example, the control unit 5063 generates a control signal for controlling the driving of the camera head 5005. At this time, in a case where the imaging condition is input by the user, the control unit 5063 generates the control signal on the basis of the input from the user. Alternatively, in a case where the endoscope 5001 has the AE function, the AF function, and the AWB function, the control unit 5063 appropriately calculates an optimum exposure value, focal length, and white balance according to a result of the wave detection processing performed by the image processing unit 5061, and generates the control signal.

Furthermore, the control unit 5063 causes the display device 5041 to display the image of the surgical site on the basis of the image signal subjected to the image processing by the image processing unit 5061. At this time, the control unit 5063 recognizes various objects in the image of the surgical site by using various image recognition technologies. For example, the control unit 5063 can recognize the surgical tool such as forceps, a specific site in the living body, bleeding, mist at the time of using the energy treatment tool 5021, and the like by detecting an edge shape, color, and the like of the object included in the image of the surgical site. When displaying the image of the surgical site on the display device 5041, the control unit 5063 superimposes various types of surgery support information on the image of the surgical site by using the recognition result. The surgery support information is superimposed and presented to the operator 5067, such that the surgery can be more safely and reliably performed.

The transmission cable 5065 connecting the camera head 5005 and the CCU 5039 is an electric signal cable supporting electric signal communication, an optical fiber supporting optical communication, or a composite cable thereof.

Here, in the illustrated example, wired communication is performed using the transmission cable 5065, but wireless communication may be performed between the camera head 5005 and the CCU 5039. In a case where wireless communication is performed between the camera head 5005 and the CCU 5039, it is not necessary to install the transmission cable 5065 in the operating room, and thus, a situation in which movement of a medical staff in the operating room is hindered by the transmission cable 5065 can be eliminated.

Hereinabove, an example of the endoscopic surgery system 5000 to which the technology according to the present disclosure can be applied has been described. Note that, here, the endoscopic surgery system 5000 has been described as an example, but a system to which the technology according to the present disclosure can be applied is not limited to such an example. For example, the technology according to the present disclosure may be applied to a flexible endoscope system for examination or a microscopic surgery system.

<2-2. Specific Configuration Example of Support Arm Device>

The medical system of the present embodiment includes a support arm device. Hereinafter, a specific configuration example of the support arm device according to an embodiment of the present disclosure will be described in detail. Note that the use of the support arm device as described below is not limited to medical use.

The support arm device described below is an example configured as a support arm device that supports an endoscope at a distal end of an arm portion, but the present embodiment is not limited to such an example. Furthermore, in a case where the support arm device according to the embodiment of the present disclosure is applied to the medical field, the support arm device according to the embodiment of the present disclosure can function as a medical support arm device.

Note that the support arm device described below can not only be applied to the endoscopic surgery system 5000, but also be applied to other medical systems. It is a matter of course that the support arm device described below can also be applied to a system other than medical systems. Furthermore, as a control unit (control device) that performs processing of the present embodiment is installed in the support arm device, the support arm device itself may be regarded as the medical system of the present embodiment.

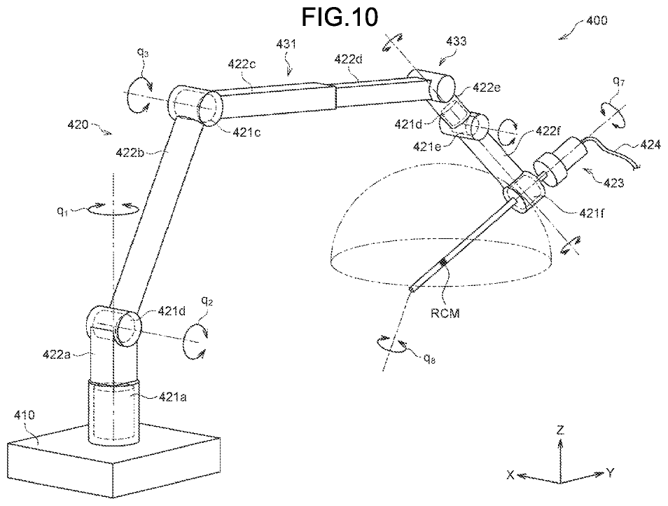

FIG. 10 is a schematic diagram illustrating an appearance of a support arm device 400 according to the present embodiment. The support arm device 400 corresponds to, for example, the robot arm A illustrated in FIGS. 1 to 3 and 5. Hereinafter, a schematic configuration of the support arm device 400 according to the present embodiment will be described with reference to FIG. 10.

The support arm device 400 according to the present embodiment includes a base portion 410 and an arm portion 420. The base portion 410 is a base of the support arm device 400, and the arm portion 420 extends from the base portion 410. Furthermore, although not illustrated in FIG. 10, a control unit that comprehensively controls the support arm device 400 may be provided in the base portion 410, and driving of the arm portion 420 may be controlled by the control unit. The control unit is implemented by, for example, various signal processing circuits such as a CPU and a digital signal processor (DSP).

The arm portion 420 includes a plurality of active joint portions 421a to 421f, a plurality of links 422a to 422f, and an endoscope device 423 as a distal end unit provided at a distal end of the arm portion 420.

The links 422a to 422f are substantially rod-shaped members. One end of the link 422a is connected to the base portion 410 via the active joint portion 421a, the other end of the link 422a is connected to one end of the link 422b via the active joint portion 421b, and the other end of the link 422b is connected to one end of the link 422c via the active joint portion 421c. The other end of the link 422c is connected to the link 422d via a passive slide mechanism 431, and the other end of the link 422d is connected to one end of the link 422e via a passive joint portion 433. The other end of the link 422e is connected to one end of the link 422f via the active joint portions 421d and 421e. The endoscope device 423 is connected to the distal end of the arm portion 420, that is, the other end of the link 422f via the active joint portion 421f. In this manner, the ends of the plurality of links 422a to 422f are connected to each other by the active joint portions 421a to 421f, the passive slide mechanism 431, and the passive joint portion 433 with the base portion 410 as a fulcrum, thereby forming an arm shape extending from the base portion 410.

A position and a posture of the endoscope device 423 are controlled by performing a control of driving actuators provided in the active joint portions 421a to 421f of the arm portion 420. In the present embodiment, a distal end of the endoscope device 423 enters the body cavity of the patient, which is the surgical site, and captures an image of a partial region of the surgical site. However, the distal end unit provided at the distal end of the arm portion 420 is not limited to the endoscope device 423, and various medical instruments may be connected to the distal end of the arm portion 420 as the distal end unit. As described above, the support arm device 400 according to the present embodiment is configured as a medical support arm device including a medical instrument.

Hereinafter, coordinate axes are defined as illustrated in FIG. 10 to describe the support arm device 400. Further, a top-bottom direction, a front-rear direction, and a left-right direction are defined in accordance with the coordinate axes. That is, a top-bottom direction with respect to the base portion 410 installed on a floor surface is defined as a z-axis direction and the top-bottom direction. Furthermore, a direction which is orthogonal to a z axis and in which the arm portion 420 extends from the base portion 410 (that is, a direction in which the endoscope device 423 is positioned with respect to the base portion 410) is defined as a y-axis direction and the front-rear direction. Further, a direction orthogonal to a y axis and the z axis are defined as an x-axis direction and the left-right direction.

The active joint portions 421a to 421f rotatably connect the links to each other. The active joint portions 421a to 421f each have actuators, and have a rotation mechanism that is rotated with respect to a predetermined rotation axis by driving of the actuators. It is possible to control the driving of the arm portion 420 such as extending or contracting (folding) of the arm portion 420 by controlling the rotation of each of the active joint portions 421a to 421f. Here, the driving of the active joint portions 421a to 421f can be controlled by a known whole body cooperative control and ideal joint control, for example. As described above, since the active joint portions 421a to 421f each have the rotation mechanism, in the following description, a driving control for the active joint portions 421a to 421f specifically means that rotation angles and/or generated torques (torques generated by the active joint portions 421a to 421f ) of the active joint portions 421a to 421f are controlled.

The passive slide mechanism 431 is an aspect of a passive form change mechanism, and connects the link 422c and the link 422d to each other so as to be movable forward and backward along a predetermined direction. For example, the passive slide mechanism 431 may connect the link 422c and the link 422d to each other so as to be linearly movable. However, a forward and backward motion of the link 422c and the link 422d is not limited to a linear motion, and may be a forward and backward motion in a direction forming an arc shape. For example, the user moves the passive slide mechanism 100 forward and backward, such that a distance between the active joint portion 421c on one end side of the link 422c and the passive joint portion 433 varies. As a result, the overall form of the arm portion 420 can be changed.

The passive joint portion 433 is an aspect of the passive form change mechanism, and rotatably connects the link 422d and the link 422e to each other. For example, the user rotates the passive joint portion 433, such that an angle formed by the link 422d and the link 422e varies. As a result, the overall form of the arm portion 420 can be changed.

The support arm device 400 according to the present embodiment includes six active joint portions 421a to 421f, and six degrees of freedom are implemented when the arm portion 420 is driven. That is, while a driving control for the support arm device 400 is implemented by a driving control for the six active joint portions 421a to 421f by the control unit, the passive slide mechanism 431 and the passive joint portion 433 are not targets of a driving control performed by the control unit.

Specifically, as illustrated in FIG. 10, the active joint portions 421a, 421d, and 421f are provided so as to have, as rotation axis directions, a major axis direction of each of the connected links 422a and 422e and an imaging direction of the connected endoscope device 423. The active joint portions 421b, 421c, and 421e are provided so as to have, as the rotation axis direction, the x-axis direction which is a direction in which a connection angle of each of the connected links 422a to 422c, 422e, and 422f and the endoscope device 423 is changed in a y-z plane (a plane defined by the y axis and the z axis). As described above, in the present embodiment, the active joint portions 421a, 421d, and 421f have a function of performing so-called yawing, and the active joint portions 421b, 421c, and 421e have a function of performing so-called pitching.

With such a configuration of the arm portion 420, in the support arm device 400 according to the present embodiment, six degrees of freedom is implemented when the arm portion 420 is driven, and thus, the endoscope device 423 can be freely moved within a movable range of the arm portion 420. In FIG. 10, a hemisphere is illustrated as an example of the movable range of the endoscope device 423. Assuming that a central point of the hemisphere, remote center of motion (RCM), is the center of the image of the surgical site captured by the endoscope device 423, the image of the surgical site can be captured at various angles by moving the endoscope device 423 on a spherical surface of the hemisphere in a state in which the center of the image captured by the endoscope device 423 is fixed to the central point of the hemisphere.

The schematic configuration of the support arm device 400 according to the present embodiment has been described above. Next, the whole body cooperative control and the ideal joint control for controlling the driving of the arm portion 420 in the support arm device 400 according to the present embodiment, that is, the driving of the active joint portions 421a to 421f, will be described.

Note that, although a case where the arm portion 220 of the support arm device 400 has a plurality of joint portions and has six degrees of freedom has been described, the present disclosure is not limited thereto. Specifically, the arm portion 220 may have a structure in which the endoscope device 423 or an exoscope is provided at the distal end. For example, the arm portion 220 may have a configuration having only one degree of freedom with which the endoscope device 423 is driven to move in a direction in which the endoscope device enters the body cavity of the patient and a direction in which the endoscope device moves backward.

<2-3. Specific Configuration Example of Endoscope>

An endoscope can be installed in the support arm device of the present embodiment. Hereinafter, a basic configuration of an oblique-viewing endoscope will be described as an example of the endoscope of the present embodiment. Note that the endoscope of the present embodiment is not limited to the oblique-viewing endoscope described below as long as a direction of an objective lens is inclined (or can be tilted) with respect to an axial direction of a main body of the endoscope.

FIG. 11 is a schematic diagram illustrating a configuration of an oblique-viewing endoscope 4100 according to an embodiment of the present disclosure. As illustrated in FIG. 11, the oblique-viewing endoscope 4100 is attached to a distal end of a camera head 4200. The oblique-viewing endoscope 4100 corresponds to the lens barrel 5003 described with reference to FIG. 8, and the camera head 4200 corresponds to the camera head 5005 described with reference to FIGS. 8 and 9. Note that the endoscope 5001 illustrated in FIG. 8 may be regarded as the oblique-viewing endoscope 4100.

The oblique-viewing endoscope 4100 and the camera head 4200 are rotatable independently of each other. An actuator is provided between the oblique-viewing endoscope 4100 and the camera head 4200 similarly to each of the joint portions 5033a, 5033b, and 5033c, and the oblique-viewing endoscope 4100 rotates with respect to the camera head 4200 by driving of the actuator.

The oblique-viewing endoscope 4100 is supported by the support arm device 5027. The support arm device 5027 has a function of holding the oblique-viewing endoscope 4100 instead of the scopist and moving the oblique-viewing endoscope 4100 so that a desired site can be observed according to an operation performed by the operator or the assistant.

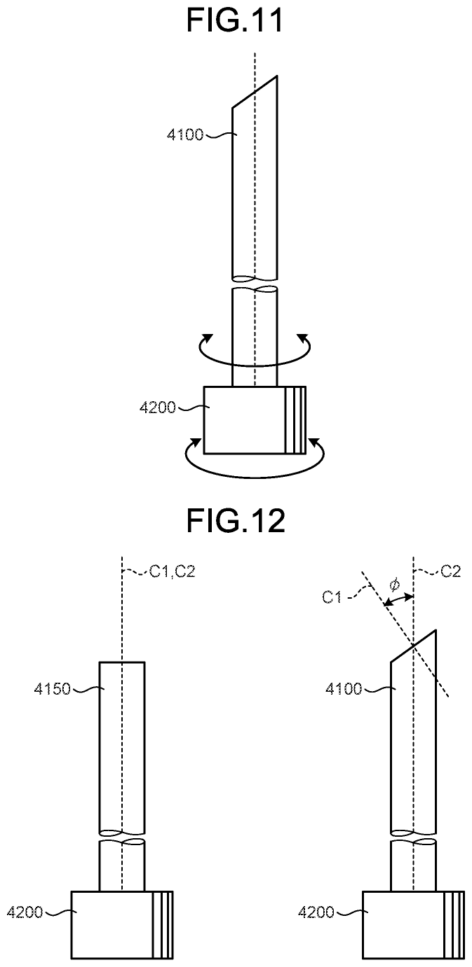

FIG. 12 is a schematic view illustrating the oblique-viewing endoscope 4100 and a forward-viewing endoscope 4150 in comparison. In the forward-viewing endoscope 4150, an orientation (C1) of the objective lens toward a subject coincides with a longitudinal direction (C2) of the forward-viewing endoscope 4150. On the other hand, in the oblique-viewing endoscope 4100, a predetermined angle ϕ is formed between the orientation (C1) of the objective lens toward the subject and the longitudinal direction (C2) of the oblique-viewing endoscope 4100. Note that in a case where the angle ϕ is 90 degrees, the oblique-viewing endoscope 4100 is called a side-viewing endoscope.

<2-4. Second Configuration Example (Medical Observation System)>

Next, a configuration of a medical observation system 1 will be described as another configuration example of the medical system of the present embodiment. Note that the support arm device 400 and the oblique-viewing endoscope 4100 described above can also be applied to the medical observation system described below. In addition, the medical observation system described below may be regarded as a functional configuration example or a modification of the endoscopic surgery system 5000 described above.

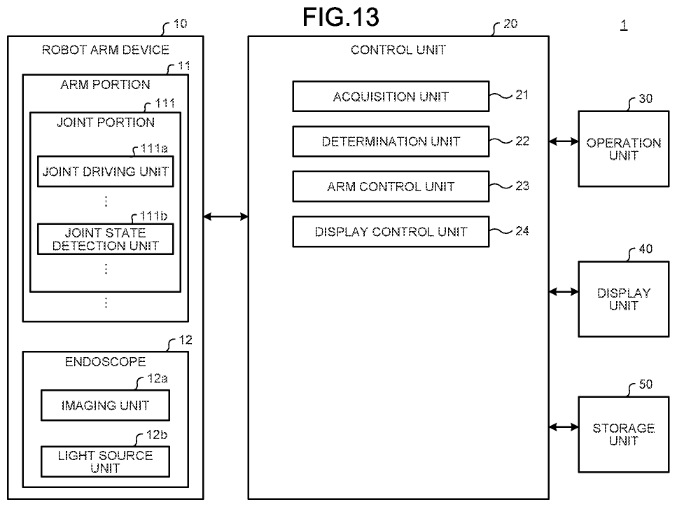

FIG. 13 is a block diagram illustrating an example of a configuration of the medical observation system 1 according to an embodiment of the present disclosure. Hereinafter, a configuration of the medical observation system according to the embodiment of the present disclosure will be described with reference to FIG. 13.

As illustrated in FIG. 13, the medical observation system 1 includes a robot arm device 10, a control unit 20, an operation unit 30, and a display unit 40.

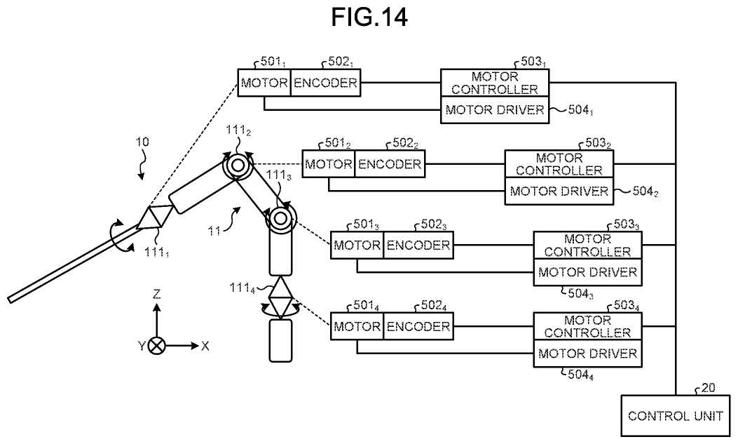

FIG. 14 is a diagram illustrating a specific configuration example of the robot arm device 10 according to the embodiment of the present disclosure. The robot arm device 10 includes, for example, an arm portion 11 (articulated arm) that is a multilink structure including a plurality of joint portions and a plurality of links. The robot arm device 10 corresponds to, for example, the robot arm A illustrated in FIGS. 1 to 3 and 5 or the support arm device 400 illustrated in FIG. 10. The robot arm device 10 is operated under the control of the control unit 20. The robot arm device 10 controls a position and a posture of a distal end unit (for example, an endoscope) provided at a distal end of the arm portion 11 by driving the arm portion 11 within a movable range. The arm portion 11 corresponds to, for example, the arm portion 420 illustrated in FIG. 10.

The arm portion 11 includes a plurality of joint portions 111. FIG. 13 illustrates a configuration of one joint portion 111 as a representative of the plurality of joint portions.

The joint portion 111 rotatably connects the links in the arm portion 11, and rotation thereof is controlled under the control of the control unit 20, thereby driving the arm portion 11. The joint portions 111 correspond to, for example, the active joint portions 421a to 421f illustrated in FIG. 10. Furthermore, the joint portion 111 may have an actuator.

As illustrated in FIG. 13, the joint portion 111 includes one or more joint driving units 111a and one or more joint state detection units 111b.

The joint driving unit 111a is a driving mechanism in the actuator of the joint portion 111, and the joint driving unit 111a performs driving to rotate the joint portion 111. The joint driving unit 111a corresponds to a motor 501₁ illustrated in FIG. 14 and the like. The driving of the joint driving unit 111a is controlled by an arm control unit 25. For example, the joint driving unit 111a corresponds to a motor and a motor driver. The driving performed by the joint driving unit 111a corresponds to, for example, driving the motor by the motor driver with a current amount according to a command from the control unit 20.

The joint state detection unit 111b is, for example, a sensor that detects a state of the joint portion 111. Here, the state of the joint portion 111 may mean a state of a motion of the joint portion 111. For example, the state of the joint portion 111 includes information such as a rotation angle, a rotation angular speed, a rotation angular acceleration, and a generated torque of the joint portion 111. The joint state detection unit 111b corresponds to an encoder 502₁ and the like illustrated in FIG. 14. In the present embodiment, the joint state detection unit 111b functions as, for example, a rotation angle detection unit that detects the rotation angle of the joint portion 111 and a torque detection unit that detects the generated torque of the joint portion 111 and an external torque. Note that the rotation angle detection unit and the torque detection unit may be an encoder and a torque sensor of the actuator, respectively. The joint state detection unit 111b transmits the detected state of the joint portion 111 to the control unit 20.

Returning to FIG. 13, the robot arm device 10 includes an endoscope 12 in addition to the arm portion 11. The endoscope 12 is, for example, an oblique-viewing endoscope. The endoscope 12 corresponds to, for example, the oblique-viewing endoscope E illustrated in FIGS. 1 to 3 and 5, the endoscope 5001 illustrated in FIG. 8, or the oblique-viewing endoscope 4100 illustrated in FIG. 11. The endoscope 12 is detachably provided at the distal end of the arm portion 11, for example. As illustrated in FIG. 13, the endoscope 12 includes an imaging unit 12a and a light source unit 12b.

The imaging unit 12a captures images of various imaging targets. The imaging unit 12a captures, for example, an operative field image including various medical instruments, organs, and the like in the abdominal cavity of the patient. Specifically, the imaging unit 12 is a camera or the like capable of capturing an image of the imaging target in a form of a moving image or a still image. More specifically, the imaging unit 12a is a wide-angle camera including a wide-angle optical system. That is, the operative field image is an operative field image captured by the wide-angle camera. For example, although an angle of view of a normal endoscope is about 80°, an angle of view of the imaging unit 12 according to the present embodiment may be 140°. Note that the angle of view of the imaging unit 12a may be greater than 80° and less than 140°, or may be equal to or greater than 140°. The imaging unit 12a transmits an electric signal (image signal) corresponding to the captured image to the control unit 20. Note that, in FIG. 13, the imaging unit 12a does not need to be included in the robot arm device, and an aspect thereof is not limited as long as the imaging unit 12a is supported by the arm portion 11.

In the light source unit 12b, the imaging unit 12a irradiates the imaging target with light. The light source unit 12b can be implemented by, for example, a wide-angle lens LED. For example, the light source unit 12b may be implemented by combining a normal LED and a lens to diffuse light. In addition, the light source unit 12b may be configured to diffuse (increase the angle of) light transmitted through an optical fiber with a lens. Further, the light source unit 12b may expand an irradiation range by irradiating the optical fiber itself with light in a plurality of directions. Note that, in FIG. 8, the light source unit 12b does not need to be included in the robot arm device 10, and an aspect thereof is not limited as long as the irradiation light can be guided to the imaging unit 12a supported by the arm portion 11.

Next, a specific configuration example of the robot arm device 10 according to the embodiment of the present disclosure will be described with reference to FIG. 14.

For example, as illustrated in FIG. 14, the arm portion 11 of the robot arm device 10 includes a first joint portion 111₁, a second joint portion 111₂, a third joint portion 111₃, and a fourth joint portion 111₄.

The first joint portion 111₁ includes the motor 501₁, the encoder 502₁, a motor controller 503₁, and a motor driver 504₁. Since the second joint portion 111₂ to the fourth joint portion 111₄ also have the same configuration as the first joint portion 111₁, the first joint portion 111₁ will be described below as an example.

Note that each of the joint portions including the first joint portion 111₁ may include a brake of the motor 501. At this time, the brake may be a mechanical brake. Then, the joint portion may be configured to maintain a current state of the arm portion 11 by using the brake, for example, in a case where the motor is not operated. Even in a case where supply of power to the motor is stopped for some reason, since the arm portion 11 is fixed by the mechanical brake, the endoscope does not move to an unintended position.

The motor 5011 is driven under the control of the motor driver 504₁ to drive the first joint portion 111₁. The motor 501₁ and/or the motor driver 504₁ corresponds to, for example, the joint driving unit 111a illustrated in FIG. 11. The motor 501₁ drives the first joint portion 111₁ in a direction of an arrow attached to the first joint portion 111₁, for example. The motor 501₁ controls the position and the posture of the arm portion 11 or positions and postures of the lens barrel and the camera by driving the first joint portion 111₁. Note that, in the present embodiment, as one form of the endoscope, a camera (for example, the imaging unit 12) may be provided at a distal end of a lens barrel.

The encoder 502₁ detects information regarding a rotation angle of the first joint portion 111₁ under the control of the motor controller 503₁. That is, the encoder 502₁ acquires information regarding the posture of the first joint portion 111₁. The encoder 502₁ detects information regarding a torque of the motor under the control of the motor controller 5031.

The control unit 20 controls the position and the posture of the arm portion 11. Specifically, the control unit 20 controls the motor controllers 5031 to 5034, the motor drivers 5041 to 5044, and the like to control the first joint portion 1111 to the fourth joint portion 1114. By doing so, the control unit 20 controls the position and the posture of the arm portion 11. The control unit 20 may be included in the robot arm device 10 or may be a device separate from the robot arm device 10. The control unit 20 corresponds to, for example, the control device that controls the robot arm A illustrated in FIGS. 1 to 3 and 5. Alternatively, the control unit 20 corresponds to, for example, the CCU 5039 or the arm control device 5045 illustrated in FIG. 8.

The control unit 20 is implemented by, for example, a central processing unit (CPU), a micro processing unit (MPU), or the like executing a program (for example, a program according to the present invention) stored in a storage unit (not illustrated) with a random access memory (RAM) or the like as a work area. Further, the control unit 20 is a controller and may be implemented by, for example, an integrated circuit such as an application specific integrated circuit (ASIC) or a field programmable gate array (FPGA).

As illustrated in FIG. 13, the control unit 20 includes an acquisition unit 21, a determination unit 22, an arm control unit 23, and a display control unit 24. The respective blocks (the acquisition unit 21, the display control unit 24, and the like) included in the control unit 20 are functional blocks each indicating the function of the control unit 20. These functional blocks may be software blocks or hardware blocks. For example, each of the above-described functional blocks may be one software module implemented by software (including a microprogram) or may be one circuit block on a semiconductor chip (die). It is a matter of course that each functional block may be one processor or one integrated circuit. A method of configuring the functional block is arbitrary. Note that the control unit 20 may be configured with a functional unit different from the above-described functional block.

For example, the acquisition unit 21 acquires an instruction from a user (for example, the operator or a person assisting the operator) who operates the operation unit 30. For example, the acquisition unit 21 acquires information regarding a situation of the surgery (for example, information regarding a currently performed treatment).

The determination unit 22 determines a combination of a plurality of operation amounts of interference avoidance operations. For example, the determination unit 22 determines a combination of an operation amount of a first interference avoidance operation and an operation amount of a second interference avoidance operation. Here, the first interference avoidance operation is, for example, the removal operation of moving the oblique-viewing endoscope so as to move the objective lens of the oblique-viewing endoscope away from the observation point. In addition, the second interference avoidance operation is, for example, the rotation operation of moving the oblique-viewing endoscope so as to change the observation direction for the observation point.

The determination unit 22 may be configured to determine a combination of an operation amount of the removal operation and an operation amount of the rotation operation. For example, the determination unit 22 may determine a combination of the operation amount of the removal operation and the operation amount of the rotation operation on the basis of a ratio between a minimum operation amount of the removal operation in a case where an interference with the surgical tool is avoided only by the removal operation and a minimum operation amount of the rotation operation in a case where an interference with the surgical tool is avoided only by the rotation operation. More specifically, the determination unit 22 may determine the combination of the operation amount of the removal operation and the operation amount of the rotation operation by calculating the ratio in a predetermined interference avoidance operation and applying the calculated ratio to design information in which a relationship between an arbitrary ratio and a combination that enables interference avoidance at the arbitrary ratio is recorded.

Here, the design information may be information of a program diagram (for example, information of the designed line as illustrated in FIG. 7) in which a first axis represents the operation amount of the removal operation and a second axis orthogonal to the first axis represents the operation amount of the rotation operation. Then, the determination unit 22 may determine the combination of the operation amount of the removal operation and the operation amount of the rotation operation by using different design information for each treatment performed by the operator.

Note that the treatment performed by the operator may include at least a first treatment and a second treatment required to be more precise than the first treatment. The design information may include first design information and second design information designed so that the operation amount of the removal operation is smaller than that of the first design information in at least some cases. At this time, in a case where the current treatment is the first treatment, the determination unit 22 may determine the combination of the operation amount of the removal operation and the operation amount of the rotation operation on the basis of the first design information. Furthermore, in a case where the current treatment is the second treatment, the determination unit 22 may determine the combination of the operation amount of the removal operation and the operation amount of the rotation operation on the basis of the second design information.

Note that the treatment performed by the operator may include at least one of a treatment of sucking liquid in the body, a treatment of clipping a blood vessel, a suturing treatment, dissection processing, or discission processing. For example, the first treatment described above may be the treatment of sucking liquid in the body. The second treatment described above may also be the treatment of clipping a blood vessel.

In addition, the treatment performed by the operator may include at least the discission processing. Then, the determination unit 22 may determine a different combination for each of a timing at which the operator pinches a tissue with the surgical tool for discission and a timing at which discission is performed.

Note that information for selecting the design information is not limited to the information regarding the treatment. For example, the determination unit 22 may determine the combination of the operation amount of the removal operation and the operation amount of the rotation operation by using the design information selected on the basis of information regarding a size of the working space (for example, information regarding a size of an area around the site to be treated by the operator).

The arm control unit 23 comprehensively controls the robot arm device 10 and controls the driving of the arm portion 11. Specifically, the arm control unit 25 controls the driving of the arm portion 11 by controlling the driving of the joint portion 111. More specifically, the arm control unit 25 controls a rotation speed of the motor by controlling the amount of current supplied to the motor in the actuator of the joint portion 111, thereby controlling the rotation angle and the generated torque of the joint portion 111.

The arm control unit 23 can cause the support arm to perform a plurality of different interference avoidance operations for avoiding an interference between the oblique-viewing endoscope and the surgical tool while maintaining a state in which the objective lens of the oblique-viewing endoscope is directed to the observation point. For example, the arm control unit 23 can cause the support arm to perform, as the interference avoidance operations, the first interference avoidance operation and the second interference avoidance operation different from the first interference avoidance operation. Here, the first interference avoidance operation is, for example, the removal operation of moving the oblique-viewing endoscope so as to move the objective lens of the oblique-viewing endoscope away from the observation point. In addition, the second interference avoidance operation is, for example, the rotation operation of moving the oblique-viewing endoscope so as to change the observation direction for the observation point.

The display control unit 24 causes the display unit 40 to display various images (including not only still images but also videos). For example, the display control unit 24 causes the display unit 40 to display the image captured by the imaging unit 12.

The operation unit 30 receives various types of operation information from the user. The operation unit 30 is implemented by, for example, a microphone that detects a voice, a gaze sensor that detects a gaze, a switch that receives a physical operation, or a touch panel. The operation unit 30 may be implemented by other physical mechanisms.

The display unit 40 displays various images. The display unit 40 is, for example, a display. For example, the display unit 40 may be a display such as a liquid crystal display (LCD) or an organic electroluminescence (EL) display. For example, the display unit 40 displays an image captured by the imaging unit 12.

A storage unit 50 is a data readable/writable storage device such as a dynamic random access memory (DRAM), a static random access memory (SRAM), a flash memory, or a hard disk. The storage unit 50 stores information of the program diagram. Here, the information of the program diagram may be, for example, as illustrated in FIG. 7, design information designed so that the first axis (for example, a vertical axis) represents the operation amount of the removal operation, and the second axis (for example, a horizontal axis) orthogonal to the first axis represents the operation amount of the rotation operation.

A plurality of pieces of design information may be recorded in the storage unit 50. For example, different design information may be recorded in the storage unit 50 for each treatment performed by the operator. At this time, the storage unit 50 may include the first design information (for example, the design information of “suction” illustrated in FIG. 7) and the second design information (for example, the design information of “clipping” illustrated in FIG. 7) designed so that the operation amount of the removal operation is smaller than that of the first design information in at least some cases.

A treatment targeted by the design information is not limited to suction and clipping, and may be the suturing treatment, the dissection treatment, or the discission treatment.

In a case of the suturing treatment, it is desirable that observation can be performed at a certain magnification to some extent such that a position through which a needle passes can be finely adjusted even though a direction from which the site to be treated is viewed is slightly changed. Therefore, it is desirable that the designer designs the design information of the suturing processing so that the operation amount of the removal operation is smaller than that of predetermined design information (for example, the design information of the suction treatment) in at least some cases.

The dissection processing has an observation requirement similar to that of the suturing processing. However, in the dissection processing, the magnification is relatively less important than in the suturing processing. Therefore, the designer may design the design information of the dissection processing so that the operation amount of the removal operation is larger than that of the design information of the suturing treatment in at least some cases.

Two timings including the timing at which the operator pinches the tissue with the surgical tool for discission and the timing at which discission is performed can be assumed in the discission processing. The designer may design different design information for each of the timing at which the operator pinches the tissue with the surgical tool for discission and the timing at which discission is performed.

In general, it is assumed that the operator focuses on observation under magnification at the timing at which the operator pinches the tissue with the surgical tool for discission. Therefore, it is desirable that the designer designs the design information so that the avoidance operation using the rotation of the oblique-viewing endoscope is actively selected rather than the removal operation at the timing at which the operator pinches the tissue with the surgical tool for discission.

On the other hand, it is assumed that the operator desires to greatly zoom out a screen to perform a work at the timing at which discission is performed. Therefore, it is desirable that the designer designs the design information so that interference avoidance using the removal operation rather than the rotation operation is actively selected at the timing at which discission is performed.

Note that the design information is not limited to information for each treatment. For example, the storage unit 50 may store the design information for each size of the working space. For example, the storage unit 50 may store the design information for each size (for example, each certain size level) of the area around the site to be treated by the operator.

For example, in a case where there are many organs such as the stomach and the liver in the periphery and the working space is small as in treatment of the pancreas, it is difficult to achieve avoidance by using the rotation operation. Therefore, the designer designs the design information so that the degree of insertion/removal is relatively high. On the other hand, in a case where a relatively large space is likely to be secured in the periphery as in treatment of the gallbladder, the designer designs the design information so that the operation amount of the rotation operation is larger as compared with a treatment in a small space (for example, the treatment of the pancreas).

Note that, in this example, the design information is divided on the basis of an organ to be treated. The design information may be divided only on the basis of the size of the space, regardless of the organ to be treated. In this case, the acquisition unit 21 of the control unit 20 may acquire a distance to a peripheral organ or tissue from a time-of-flight (ToF) sensor or a stereo image sensor, or an image information processing result. Then, the determination unit 22 of the control unit 20 may select the design information for determining the combined operation amount on the basis of the size of the space instead of the organ to be treated.

<<3. Operation of Medical System>>

The configuration of the medical system has been described above, and the operation of the medical system will be described below. In the following description, an example in which a support arm that supports an oblique-viewing endoscope is controlled will be described.

Note that although it is assumed in the following description that the medical system of the present embodiment is the medical observation system 1, the operation described below can be applied not only to the medical observation system 1 but also to other medical systems.

The medical observation system 1 autonomously performs the interference avoidance operation for the oblique-viewing endoscope and the surgical tool. As described above, the interference avoidance operation is determined according to the combination of the removal operation of pulling the oblique-viewing endoscope and the rotation operation of rotating the oblique-viewing endoscope. The control unit 20 included in the medical observation system 1 determines the combined operation amount of the removal operation and the rotation operation of the oblique-viewing endoscope on the basis of the R/I ratio and the information of the program diagram designed in advance.

The R/I ratio is the ratio between the minimum operation amount of the removal operation in a case where an interference with the surgical tool is avoided only by the removal operation and the minimum operation amount of the rotation operation in a case where an interference with the surgical tool is avoided only by the rotation operation. In the following description, it is assumed that information (for example, the design information of “suction” illustrated in FIG. 7 and the design information of “clipping” illustrated in FIG. 7) of a plurality of program diagrams designed in advance is recorded in the storage unit 50 of the medical observation system 1.

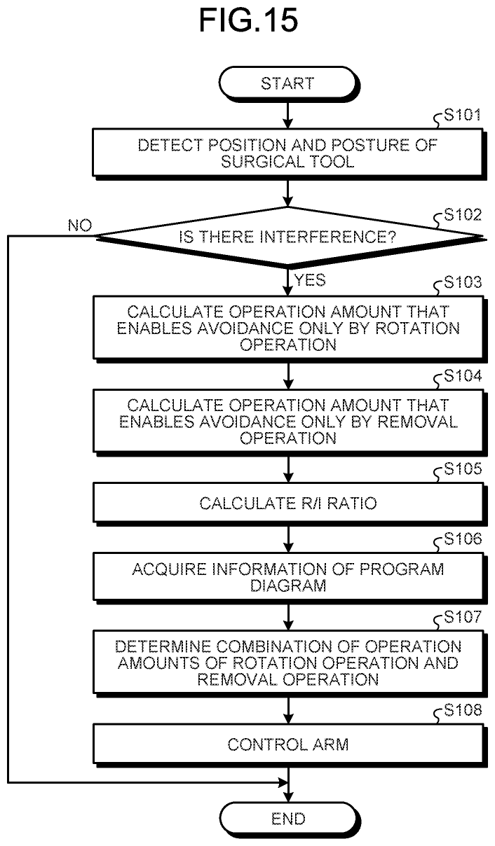

FIG. 15 is a flowchart illustrating an example of interference avoidance processing for avoiding an interference between the oblique-viewing endoscope and the surgical tool. Hereinafter, control processing according to an embodiment of the present invention will be described with reference to FIG. 15.

First, the control unit 20 detects the position of the surgical tool and the posture of the endoscope 12 on the basis of the image captured by the endoscope 12 (Step S101). As described above, the endoscope 12 is an oblique-viewing endoscope.

Then, the control unit 20 determines whether or not the endoscope 12 and the surgical tool interfere with each other (Step S102). For example, as illustrated in FIG. 4, for example, the control unit 20 determines whether or not a distal end portion of the endoscope 12 (the oblique-viewing endoscope E in the example of FIG. 5) is positioned inside the interference avoidance area set in a columnar shape around the surgical tool (the surgical tool S1 in the example of FIG. 4), for example, as illustrated in FIG. 5. In a case where there is no interference (Step S102: No), the control unit 20 ends the processing.

In a case where there is an interference (Step S102: Yes), the control unit 20 calculates a minimum operation amount (rotation amount) of the rotation operation that enables avoidance of an interference with the surgical tool only by the rotation operation (Step S103). This operation amount is, for example, the rotation amount θ in the example of FIG. 6. rθ may be an operation amount calculated in Step S103. Here, r is a radius of a circle formed by cutting the cone along the rotation direction R so as to pass through the current position P in the example of FIG. 5.

Subsequently, the control unit 20 calculates the minimum operation amount (the degree of insertion/removal) of the rotation operation that enables avoidance of an interference with the surgical tool only by the removal operation (Step S104). This operation amount is, for example, the degree L of insertion/removal in the example of FIG. 6.

Subsequently, the control unit 20 calculates the R/I ratio on the basis of the rotation amount calculated in Step S103 and the degree of insertion/removal calculated in Step S104 (Step S105). As described above, the R/I ratio is the ratio between the minimum operation amount of the removal operation in a case where an interference with the surgical tool is avoided only by the removal operation and the minimum operation amount of the rotation operation in a case where an interference with the surgical tool is avoided only by the rotation operation. For example, the control unit 20 calculates the R/I ratio on the basis of Equation (1) or Equation (2) described in <1-1. Purpose and the like of Present Embodiment>.

Subsequently, the control unit 20 acquires the information of the program diagram from the storage unit 50 (Step S106). The information of the program diagram is, for example, the design information for determining the combined operation amount as illustrated in FIG. 7. At this time, the control unit 20 may select the design information for determining the combined operation amount among a plurality of pieces of design information on the basis of the information regarding the treatment performed by the operator.

Subsequently, the control unit 20 determines the combined operation amount of the rotation operation and the removal operation on the basis of the R/I ratio calculated in Step S105 and the information of the program diagram acquired in Step S106 (Step S107). For example, it is assumed that the R/I ratio calculated in Step S105 is indicated by the oblique line illustrated in FIG. 7, and the information of the program diagram acquired in Step S106 is the design information of “suction” or “clipping” illustrated in FIG. 7. At this time, in a case where the treatment currently performed by the operator is “suction”, the control unit 20 sets, as the combined operation amount, the values of R and I indicated by the intersection CP1 of the oblique line indicating the R/I ratio and the designed line indicating the suction. On the other hand, in a case where the treatment currently performed by the operator is “clipping”, the control unit 20 sets, as the combined operation amount, the values of R and I indicated by the intersection CP2 of the oblique line indicating the R/I ratio and the designed line indicating the suction. Note that the information regarding the treatment currently performed by the operator may be input to the control unit 20 by the operator or an assistant thereof via the operation unit 30, or may be discriminated by the control unit 20 from, for example, the shape of the surgical tool or the like on the basis of the image captured by the endoscope 12.

Then, the control unit 20 controls the arm portion 11 on the basis of the combined operation amount determined in Step S107 (Step S108). Once the control of the arm portion 11 is completed, the control unit 20 ends the interference avoidance processing.

As a result, the medical observation system 1 can perform an appropriate interference avoidance operation according to the situation of the surgery. For example, the medical observation system 1 can perform the interference avoidance operation in which the loss of details and the change of the rotation direction are balanced according to the treatment performed by the operator or the size of the working space in which the treatment is performed.

<<4. Modification>>

The above-described embodiments are only examples, and various modifications and applications are possible.

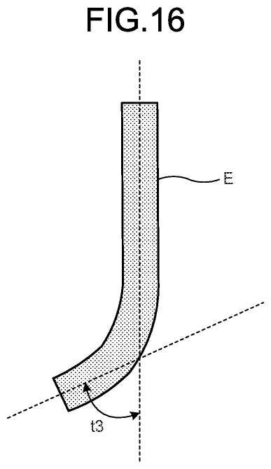

For example, in the above-described embodiments, the oblique-viewing endoscope in which the distal end portion of the shaft-shaped main body is cut obliquely with respect to the axial direction as illustrated in FIGS. 2 and 11 has been exemplified as the oblique-viewing endoscope. However, the oblique-viewing endoscope is not limited to such a shape. FIG. 16 is a diagram illustrating a modification of the oblique-viewing endoscope. For example, the oblique-viewing endoscope may have a shape in which the distal end portion is bent with respect to the axial direction. At this time, in the oblique-viewing endoscope, a bending angle t3 may be changeable according to the operation performed by the operator.

For example, in the above-described embodiments, two operations, the rotation operation and the insertion/removal operation (the removal operation or the insertion operation), are exemplified as the interference avoidance operations, but the interference avoidance operation is not limited to these two operations. For example, the interference avoidance operation does not have to be an operation of moving the distal end of the oblique-viewing endoscope on the conical surface. For example, the control device of the support arm may move the oblique-viewing endoscope out of the conical surface as long as the target observation point is included in the image. Therefore, it is easier for the control device to achieve balance between the loss of details and the change of the direction of rotation. For example, the control device can perform an operation such as maintaining details although the observation point is not positioned at the center of the image.

In addition, the interference avoidance operation is not limited to two operations, the rotation operation and the insertion/removal operation (removal operation or insertion operation). There may be three or more interference avoidance operations. The three or more interference avoidance operations may include or do not have to include the rotation operation and the insertion/removal operation. Since the number of options for the interference avoidance operation is increased, it is easier for the control device to achieve balance between the loss of details and the change of the rotation direction.

The control device (for example, the control device of the robot arm A, the CCU 5039, the arm control device 5045, or the control unit 20) that controls the support arm of the present embodiment may be implemented by a dedicated computer system or a general-purpose computer system.

For example, a program for performing the above-described control processing is stored in a computer-readable recording medium such as an optical disk, a semiconductor memory, a magnetic tape, or a flexible disk, and distributed. Then, for example, the control device is implemented by installing the program in a computer and performing the above processing. At this time, the control device may be a device (for example, a personal computer) outside the support arm (for example, a medical support arm such as the robot arm A, the support arm device 5027, the support arm device 400, or the robot arm device 10). Furthermore, the control device may be a device (for example, a processor mounted on the support arm) inside the support arm.

Further, the communication program may be stored in a disk device included in a server device on a network such as the Internet, and be downloaded to a computer. Further, the functions described above may be implemented by cooperation between an operating system (OS) and application software. In this case, the part other than the OS may be stored in a medium and distributed, or the part other than the OS may be stored in the server device and downloaded to a computer.

Further, among the respective processing described in the above-described embodiments, all or some of the processing described as being automatically performed can be manually performed. Alternatively, all or some of the processing described as being manually performed can be automatically performed by a known method. In addition, the processing procedures, specific names, information including various data and parameters illustrated in the specification and drawings can be arbitrarily changed unless otherwise specified. For example, various information illustrated in each drawing is not limited to the illustrated information.

Further, each illustrated component of each device is functionally conceptual, and does not necessarily have to be configured physically as illustrated in the drawings. That is, the specific modes of distribution/integration of the respective devices are not limited to those illustrated in the drawings. All or some of the devices can be functionally or physically distributed/integrated in any arbitrary unit, depending on various loads or the status of use.

Further, the above-described embodiments can be appropriately combined as long as the processing contents do not contradict each other. Further, the order of each step illustrated in the flowchart of the above-described embodiment can be changed as appropriate.

Furthermore, for example, the present embodiment can be implemented as any component included in the device or system, such as a processor as a system large scale integration (LSI) or the like, a module using a plurality of processors or the like, a unit using a plurality of modules or the like, a set obtained by further adding other functions to a unit, or the like (that is, some components of the device).

Note that, in the present embodiment, the system means a set of a plurality of components (devices, modules (parts), and the like), and it does not matter whether or not all the components are in the same housing. Therefore, a plurality of devices housed in separate housings and connected via a network and one device in which a plurality of modules are housed in one housing are both systems.

Furthermore, for example, the present embodiment can adopt a configuration of cloud computing in which one function is shared and processed by a plurality of devices in cooperation via a network.

<<5. Conclusion>>

The medical support arm of the present embodiment includes: the support arm that supports the oblique-viewing endoscope; the arm control unit that is configured to cause the support arm to perform a plurality of different interference avoidance operations for avoiding an interference between the oblique-viewing endoscope and the surgical tool while maintaining a state in which the objective lens of the oblique-viewing endoscope is directed to the observation point; and the determination unit that determines the combination of the operation amounts of the plurality of interference avoidance operations.

As a result, it is possible to avoid the interference by the combination of the plurality of operations instead of avoiding the interference simply by one operation, such that the medical support arm can perform the interference avoidance operation suitable for surgery.

Note that the effects described in the present specification are merely examples. The effects of the present disclosure are not limited thereto, and other effects may be obtained.

Note that the present technology can also have the following configurations.
(1)

A medical support arm comprising:

a support arm that supports an endoscope;

an arm control unit that is configured to cause the support arm to perform a plurality of different interference avoidance operations for avoiding an interference between the endoscope and a surgical tool while maintaining a state in which an objective lens of the endoscope is directed to an observation target; and

a determination unit that determines a combination of operation amounts of the plurality of interference avoidance operations.
(2)

The medical support arm according to (1), wherein the arm control unit is configured to cause the support arm to perform, as the interference avoidance operations, a first interference avoidance operation and a second interference avoidance operation different from the first interference avoidance operation, and

the determination unit determines a combination of an operation amount of the first interference avoidance operation and an operation amount of the second interference avoidance operation.
(3)

The medical support arm according to (2), wherein the first interference avoidance operation is a removal operation of moving the endoscope so as to move the objective lens of the endoscope away from the observation target,

the second interference avoidance operation is a rotation operation of moving the endoscope so as to change an observation direction for the observation target, and

the determination unit determines a combination of an operation amount of the removal operation and an operation amount of the rotation operation.
(4)

The medical support arm according to (3), wherein the determination unit determines the combination of the operation amount of the removal operation and the operation amount of the rotation operation on a basis of a ratio between a minimum operation amount of the removal operation in a case where the interference with the surgical tool is avoided only by the removal operation and a minimum operation amount of the rotation operation in a case where the interference with the surgical tool is avoided only by the rotation operation.
(5)

The medical support arm according to (4), wherein the determination unit determines the combination of the operation amount of the removal operation and the operation amount of the rotation operation by calculating the ratio in a predetermined interference avoidance operation and applying the calculated ratio to design information in which a relationship between an arbitrary ratio and the combination that enables interference avoidance at the arbitrary ratio is recorded.
(6)

The medical support arm according to (5), wherein the design information is information of a program diagram in which a first axis represents the operation amount of the removal operation and a second axis orthogonal to the first axis represents the operation amount of the rotation operation.
(7)

The medical support arm according to (5) or (6), wherein the determination unit determines the combination of the operation amount of the removal operation and the operation amount of the rotation operation by using different design information for each treatment performed by an operator.
(8)

The medical support arm according to (7), wherein the treatment performed by the operator includes at least a first treatment and a second treatment required to be more precise than the first treatment,

the design information includes first design information and second design information designed so that the operation amount of the removal operation is smaller than that of the first design information in at least some cases, and

the determination unit determines the combination of the operation amount of the removal operation and the operation amount of the rotation operation on a basis of the first design information in a case where the first treatment is performed, and the determination unit determines the combination of the operation amount of the removal operation and the operation amount of the rotation operation on a basis of the second design information in a case where the second treatment is performed.
(9)

The medical support arm according to (8), wherein the first treatment is a treatment of sucking liquid in a body, and

the second treatment is a treatment of clipping a blood vessel.
(10)

The medical support arm according to any one of (7) to (9), wherein the treatment performed by the operator includes at least one of a treatment of sucking liquid in a body, a treatment of clipping a blood vessel, a suturing treatment, dissection processing, or discission processing.
(11)

The medical support arm according to (10), wherein the treatment performed by the operator includes at least the discission processing, and

the determination unit determines a different combination for each of a timing at which the operator pinches a tissue with the surgical tool for discission and a timing at which discission is performed.
(12)

The medical support arm according to (5), wherein the determination unit determines the combination of the operation amount of the removal operation and the operation amount of the rotation operation by using the design information selected on a basis of information regarding a size of an area around a site to be treated by an operator.
(13)

A medical system comprising:

a support arm that supports an endoscope; and

a control device that controls the support arm,

wherein the control device includes:

an arm control unit that is configured to cause the support arm to perform a plurality of different interference avoidance operations for avoiding an interference between the endoscope and a surgical tool while maintaining a state in which an objective lens of the endoscope is directed to an observation target; and

a determination unit that determines a combination of operation amounts of the plurality of interference avoidance operations.
(14)

A control device that controls a support arm supporting the endoscope, the control device including:

an arm control unit that is configured to cause the support arm to perform a plurality of different interference avoidance operations for avoiding an interference between the endoscope and a surgical tool while maintaining a state in which an objective lens of the endoscope is directed to an observation target; and

a determination unit that determines a combination of operation amounts of the plurality of interference avoidance operations.
(15)

A method of controlling a support arm supporting the endoscope, the method including:

determining a combination of operation amounts of a plurality of different interference avoidance operations for avoiding an interference between the endoscope and a surgical tool while maintaining a state in which an objective lens of the endoscope is directed to an observation target; and

controlling the support arm on the basis of the combination of the operation amounts.
(16)

A program for causing a computer that controls a support arm supporting the endoscope to function as:

an arm control unit that is configured to cause the support arm to perform a plurality of different interference avoidance operations for avoiding an interference between the endoscope and a surgical tool while maintaining a state in which an objective lens of the endoscope is directed to an observation target; and

a determination unit that determines a combination of operation amounts of the plurality of interference avoidance operations.

### REFERENCE SIGNS LIST

1 MEDICAL OBSERVATION SYSTEM

10 ROBOT ARM DEVICE

11 ARM P0RTION

111 JOINT P0RTION

111a JOINT DRIVING UNIT

111b JOINT STATE DETECTION UNIT

12 ENDOSCOPE

12a IMAGING UNIT

12b LIGHT SOURCE UNIT

20 CONTROL UNIT

21 ACQUISITION UNIT

22 DETERMINATION UNIT

23 ARM CONTROL UNIT

24 DISPLAY CONTROL UNIT

30 OPERATION UNIT

40 DISPLAY UNIT

1. A medical support arm comprising:
a support arm that supports an endoscope;
an arm control unit that is configured to cause the support arm to perform a plurality of different interference avoidance operations for avoiding an interference between the endoscope and a surgical tool while maintaining a state in which an objective lens of the endoscope is directed to an observation target; and
a determination unit that determines a combination of operation amounts of the plurality of interference avoidance operations.

2. The medical support arm according to claim 1, wherein the arm control unit is configured to cause the support arm to perform, as the interference avoidance operations, a first interference avoidance operation and a second interference avoidance operation different from the first interference avoidance operation, and
the determination unit determines a combination of an operation amount of the first interference avoidance operation and an operation amount of the second interference avoidance operation.

3. The medical support arm according to claim 2, wherein the first interference avoidance operation is a removal operation of moving the endoscope so as to move the objective lens of the endoscope away from the observation target,
the second interference avoidance operation is a rotation operation of moving the endoscope so as to change an observation direction for the observation target, and
the determination unit determines a combination of an operation amount of the removal operation and an operation amount of the rotation operation.

4. The medical support arm according to claim 3, wherein the determination unit determines the combination of the operation amount of the removal operation and the operation amount of the rotation operation on a basis of a ratio between a minimum operation amount of the removal operation in a case where the interference with the surgical tool is avoided only by the removal operation and a minimum operation amount of the rotation operation in a case where the interference with the surgical tool is avoided only by the rotation operation.

5. The medical support arm according to claim 4, wherein the determination unit determines the combination of the operation amount of the removal operation and the operation amount of the rotation operation by calculating the ratio in a predetermined interference avoidance operation and applying the calculated ratio to design information in which a relationship between an arbitrary ratio and the combination that enables interference avoidance at the arbitrary ratio is recorded.

6. The medical support arm according to claim 5, wherein the design information is information of a program diagram in which a first axis represents the operation amount of the removal operation and a second axis orthogonal to the first axis represents the operation amount of the rotation operation.

7. The medical support arm according to claim 5, wherein the determination unit determines the combination of the operation amount of the removal operation and the operation amount of the rotation operation by using different design information for each treatment performed by an operator.

8. The medical support arm according to claim 7, wherein the treatment performed by the operator includes at least a first treatment and a second treatment required to be more precise than the first treatment,
the design information includes first design information and second design information designed so that the operation amount of the removal operation is smaller than that of the first design information in at least some cases, and
the determination unit determines the combination of the operation amount of the removal operation and the operation amount of the rotation operation on a basis of the first design information in a case where the first treatment is performed, and the determination unit determines the combination of the operation amount of the removal operation and the operation amount of the rotation operation on a basis of the second design information in a case where the second treatment is performed.

9. The medical support arm according to claim 8, wherein the first treatment is a treatment of sucking liquid in a body, and
the second treatment is a treatment of clipping a blood vessel.

10. The medical support arm according to claim 7, wherein the treatment performed by the operator includes at least one of a treatment of sucking liquid in a body, a treatment of clipping a blood vessel, a suturing treatment, dissection processing, or discission processing.

11. The medical support arm according to claim 10, wherein the treatment performed by the operator includes at least the discission processing, and
the determination unit determines a different combination for each of a timing at which the operator pinches a tissue with the surgical tool for discission and a timing at which discission is performed.

12. The medical support arm according to claim 5, wherein the determination unit determines the combination of the operation amount of the removal operation and the operation amount of the rotation operation by using the design information selected on a basis of information regarding a size of an area around a site to be treated by an operator.

13. A medical system comprising:
a support arm that supports an endoscope; and
a control device that controls the support arm,
wherein the control device includes:
an arm control unit that is configured to cause the support arm to perform a plurality of different interference avoidance operations for avoiding an interference between the endoscope and a surgical tool while maintaining a state in which an objective lens of the endoscope is directed to an observation target; and
a determination unit that determines a combination of operation amounts of the plurality of interference avoidance operations.
